(19) United States
(12) Patent Application Publication
(10) Pub. No.: US 2011/0268807 A1
(43) Pub. Date: Nov. 3, 2011

(54) Biodegradable Microspheres and Methods of Use Thereof

(76) Inventors: James Su, Berkeley, CA (US); Kevin Edward Healy, Moraga, CA (US); Widya Mulyasasmita, Berkeley, CA (US)

(21) Appl. No.: 13/057,256

(22) PCT Filed: Aug. 4, 2009
(86) PCT No.: PCT/US09/52732

§ 371 Date: Apr. 22, 2011

Related U.S. Application Data
(60) Provisional application No. 61/086,122, filed on Aug. 4, 2008.

(51) Int. Cl. A61K 9/14; A61K 47/48; A61K 38/02; A61K 31/7088; B82Y 5/00

(52) U.S. Cl. 424/491; 424/497; 514/1.1; 514/44 R; 514/44 A; 424/78.17; 977/773; 977/906; 977/788

(57) ABSTRACT

The present invention provides biodegradable microspheres, compositions comprising a subject biodegradable microsphere, and methods of using a subject biodegradable microsphere for delivery of an agent to a site in an individual.

### CROSS-REFERENCE

This application claims the benefit of U.S. Provisional Patent Application No. 61/086,122, filed Aug. 4, 2008, which application is incorporated herein by reference in its entirety.

### BACKGROUND

Various vehicles for delivering pharmaceutically active agents to a treatment site in an individual have been developed, including, e.g., microspheres (polymeric micelles, liposomes, etc.), natural & synthetic hydrogels (collagen, poly-N-isopropylacylamide (pNIPAAm)-based, Matrigel, etc.), and systems incorporating the two. One of the current microsphere technologies allow for sustained release of drugs for eight days or more when embedded in a polyhydroxyethylmethacrylate (pHEMA) contact lens impregnated with lidocaine. However, the pHEMA system is suitable for encapsulating hydrophobic drugs, but is unfavorable for encapsulation of hydrophilic drugs. In addition, the system requires high temperature at 60° C. for drug encapsulation and hydrogel fabrication, which poses potential problems for encapsulating bioactive factors sensitive to high temperatures.

There is a need in the art for improved microspheres with controlled release properties.

### Literature

Gou et al. (2008) Int J Pharm 359(1-2):228-233; Gulsen and Chauhan (2004) Invest Ophthalmol Vis Sci 45:2342-2347; Kim and Healy (2003) Biomacromolecules 4:1214-1223; Liu et al. (2008) J Mater Sci Mater Med 19:3365; Liu et al. (2007) J Mater Sci Mater Med 18:2205; Nukavarapu et al. (2008) Biomacromolecules 9(7):1818-25; Park and Healy (2003) Bioconjugate Chem. 14, 311-319; Stile and Healy (2001) Biomacromolecules 2, 185-194; Ruel-Gariepy et al. (2002) J. Controlled Release 82:373; Tobio et al. (1998) Pharm. Res. 15:270; Molina et al. (2001) J. Antimicr. Chemo. 47:101; Na et al. (2006) J. Antimicr. Chemo. 27:5951; Zalfen et al. (2008) Acta Biomater. 4:1788; Cascone et al. (2002) J. Mater. Sci. Mater. Med. 13:29; Cascone et al. (2002) J. Mater. Sci. Mater. Med. 13:265; Joung et al. (2007) Biomed. Mater. 2:269; Zhang et al. (2009) Acta Biomater. 5:488; Zhang et al. (2005) Biomaterials 26:3299; Stile et al. (2001) Biomacromolecules 2:185; De Faria et al. (2005) Macromolec. Symposia 229:228; Avgoustakis (2004) Curr. Drug Deliv. 1:321; Geroski et al. (2000) Invest. Ophth. Vis. Sci. 41:961; Xu et al. (2007) J. Biomed. Mater. Res. A. 81:418; WO 2006/047279; WO 97/05185; WO 03/028589; WO 2005/002625; WO 2007/123993; U.S. Patent Publication No. 2006/0188583; U.S. Pat. No. 6,632,457; U.S. Pat. No. 5,543,158; U.S. Pat. No. 5,384,333.

### SUMMARY OF THE INVENTION

The present invention provides biodegradable microspheres, compositions comprising a subject biodegradable microsphere, and methods of using a subject biodegradable microsphere for delivery of an agent to a site in an individual.

### BRIEF DESCRIPTION OF THE DRAWINGS

FIG. 1 depicts a synthesis pathway of poly(lysine-g-(lactide-b-ethylene glycol)) terpolymer.

FIGS. 2A-C depict SEC-MALLS data of pLL-mpEG reacted from lactide monomer:hydroxyl (M/OH) ratio of 40.

FIGS. 3A-C depict SEC-MALLS data of pLL-mpEG reacted from lactide monomer:hydroxyl (M/OH) ratio of 60.

FIGS. 4A and B depict eosin Y absorbance data for the CMC determination of pLL-mpEG.

FIG. 5 depicts ¹H NMR spectrum of pLL-mpEG (Mn=6850 g/mol).

FIG. 6 depicts ¹H NMR spectrum of su-pLL-mpEG.

FIG. 7 depicts ¹H NMR spectrum of pK-pLL-mpEG terpolymer.

FIG. 8 provides Table 1, which is a summary of the data.

FIG. 9 is a schematic depiction of a nanoparticle within a subject microsphere.

FIG. 10 is a schematic depiction of various embodiments of a subject microsphere.

FIG. 11A schematically depicts ocular drug delivery of atropine; FIG. 11B depicts the structure of atropine.

FIG. 12 depicts poly(N-isopropylacrylamide-co-acrylic acid) hydrogel and poly(L-lactide-m-ethylene glycol) nanoparticles.

FIG. 13 depicts optical density vs. atropine concentration for atropine solutions.

FIGS. 14A-C depict swelling capacity of hydrogel.

FIG. 15 depicts swelling variation with time and media.

FIG. 16 depicts release rate of atropine from hydrogel.

FIGS. 17A and B depict atropine release.

FIG. 18 depicts transscleral drug delivery.

FIG. 19 depicts poly(N-isopropylacrylamide-co-acrylic acid) hydrogel and poly(L-lactide-m-ethylene glycol) synthesis.

### DEFINITIONS

As used herein, the term “copolymer” describes a polymer which contains more than one type of subunit. The term encompasses polymer which include two, three, four, five, or six types of subunits.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term “polypeptide” includes fusion proteins, including, but not limited to, fusion proteins with a heterologous amino acid sequence, fusions with heterologous and homologous leader sequences, with or without N-terminal methionine residues; immunologically tagged proteins; and the like. The term “polypeptide” includes polypeptides comprising one or more of a fatty acid moiety, a lipid moiety, a sugar moiety, and a carbohydrate moiety. The term “polypeptides” includes post-translationally modified polypeptides.

As used herein, the term “label moiety” is intended to mean one or more atoms that can be specifically detected to indicate the presence of a substance to which the one or more atom is attached. A label moiety can be a primary label that is directly detectable or secondary label that can be indirectly detected, for example, via interaction with a primary label. Exemplary primary labels include, without limitation, an isotopic label such as a naturally non-abundant heavy isotope or radioactive isotope, examples of which include ¹⁴C, ¹²³I, ¹²⁴I, ¹²⁵I, ¹³¹I, ³²P, ³⁵S or ³H; optically detectable moieties such as a chromophore, luminophore, fluorophore, quantum dot or nanoparticle; electromagnetic spin label; calorimetric agent; magnetic substance; electron-rich material such as a metal; electrochemiluminescent label such as Ru(bpy)₃²⁺; moiety that can be detected based on a nuclear magnetic, paramagnetic, electrical, charge to mass, or thermal characteristic; or light scattering or plasmon resonant materials such as gold or silver particles. Fluorophores that are useful in the invention include, for example, fluorescent lanthanide complexes, including those of Europium and Terbium, fluorescein, fluorescein isothiocyanate, carboxyfluorescein (FAM), dichlorotriazinylamine fluorescein, rhodamine, tetramethylrhodamine, umbelliferone, eosin, erythrosin, coumarin, methyl-coumarins, pyrene, Malacite green, Cy3, Cy5, stilbene, Lucifer Yellow, Cascade Blue™, Texas Red, alexa dyes, dansyl chloride, phycoerythin, green fluorescent protein and its wavelength shifted variants, bodipy, and others known in the art such as those described in Haugland, Molecular Probes Handbook, (Eugene, Oreg.) 6th Edition; The Synthegen catalog (Houston, Tex.), Lakowicz, Principles of Fluorescence Spectroscopy, 2nd Ed., Plenum Press New York (1999), or WO 98/59066.

The terms “subject,” “individual,” “host,” and “patient” are used interchangeably herein to a member or members of any mammalian or non-mammalian species. Subjects and patients thus include, without limitation, humans, non-human primates, canines, felines, ungulates (e.g., equine, bovine, swine (e.g., pig)), avians, rodents (e.g., rats, mice), and other subjects. Non-human animal models, particularly mammals, e.g. a non-human primate, a murine (e.g., a mouse, a rat), lagomorpha, etc. may be used for experimental investigations.

“Treating” or “treatment” of a condition or disease includes: (1) preventing at least one symptom of the condition, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient, in combination with another agent, or alone in one or more doses, to effect such treatment for the disease. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The term “physiological conditions” is meant to encompass those conditions compatible with living cells, e.g., predominantly aqueous conditions of a temperature, pH, salinity, etc. that are compatible with living cells.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes one and more than one such excipient, diluent, carrier, and adjuvant.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a microsphere” includes a plurality of such microspheres and reference to “the active agent” includes reference to one or more active agents and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides biodegradable microspheres, and compositions (including pharmaceutical compositions) comprising same. A subject biodegradable microsphere is suitable for use in delivering an active agent to a site (e.g., a treatment site or a diagnostic site) in an individual. A subject biodegradable microsphere thus finds use in various therapeutic and diagnostic applications, which are also provided.

### Biodegradable Microspheres

The present invention provides biodegradable microspheres, and compositions (including pharmaceutical compositions) comprising same. A subject biodegradable microsphere comprises: a) a nanoparticle or a microparticle; and b) a hydrogel matrix that forms an outer layer surrounding the nanoparticle or microparticle. The nanoparticle (or microparticle) can comprise an inner core comprising: i) a hydrophobic polymer; ii) a hydrophilic polymer linked to the hydrophobic polymer, where the hydrophobic polymer and the hydrophilic polymers together form a nanoparticle or microparticle, where the hydrophobic polymer forms an inner layer of the nanoparticle or microparticle, and the hydrophilic polymer forms an outer layer of the nanoparticle or microparticle. In some embodiments, a subject microsphere comprises a detectable label.

An active agent (as described in more detail below) can be present within the hydrophobic core of the nanoparticle or microparticle. In some embodiments, an active agent is present only within the hydrophobic core, e.g., within the space created by the hydrophobic polymer. In some embodiments, an active agent is present within the hydrophobic core, and the active agent not linked to any moiety of the nanoparticle or microparticle. In other embodiments, an active agent is present within the hydrophobic core, and the active agent is linked to one or more moieties present in the hydrophobic core, e.g., the active agent is linked to a poly-L-lactide polymer. In other embodiments, the active agent is linked to the hydrophilic polymer, e.g., in some embodiments, the active agent is linked to a poly(ethylene glycol) (PEG) (e.g., the active agent is linked to a reactive group present on derivatized PEG; e.g., the active agent is linked to an amine group present on derivatized PEG).

In some embodiments, an active agent is released from the hydrogel at a rate such that from about 50% to about 100% (e.g., from about 50% to about 60%, from about 60% to about 70%, from about 70% to about 80%, from about 80% to about 90%, or from about 90% to about 100%) of the active agent is released within about 1 hour to about 48 hours (e.g., from about 1 hour to about 2 hours, from about 2 hours to about 4 hours, from about 4 hours to about 8 hours, from about 8 hours to about 12 hours, from about 12 hours to about 18 hours, from about 18 hours to about 24 hours, from about 24 hours to about 36 hours, or from about 36 hours to about 48 hours).

In some embodiments, an active agent is released from the nanoparticle (or microparticle) at a rate such that from about 50% to about 100% (e.g., from about 50% to about 60%, from about 60% to about 70%, from about 70% to about 80%, from about 80% to about 90%, or from about 90% to about 100%) of the active agent is released over a certain time period after the hydrogel degrades, e.g., the active agent is release from the nanoparticle or microparticle over a period of time of from about 1 hour to about 48 hours (e.g., from about 1 hour to about 2 hours, from about 2 hours to about 4 hours, from about 4 hours to about 8 hours, from about 8 hours to about 12 hours, from about 12 hours to about 18 hours, from about 18 hours to about 24 hours, from about 24 hours to about 36 hours, or from about 36 hours to about 48 hours), or over a period of time of from about 2 days to about 12 days (e.g., from about 2 days to about 4 days, from about 4 days to about 6 days, from about 6 days to about 8 days, from about 8 days to about 10 days, or from about 10 days to about 12 days).

In some embodiments, the hydrogel degrades over a period of time of from about 0.5 day to about 4 days, e.g., from about 50% to about 100% of the hydrogel is degraded within a period of time of from about 0.5 day to about 1 day, from about 1 day to about 2 days, or from about 2 days to about 4 days.

In some embodiments, a first active agent is present within the hydrophobic core; and a second active agent is linked to the hydrophilic polymer. Where a first active agent is present within the hydrophobic core, and a second active agent is linked to the hydrophilic polymer, the first active agent is different from the second active agent. For example, where a first active agent is present within the hydrophobic core, and a second active agent is linked to the hydrophilic polymer, the first active agent is hydrophobic, and the second active agent is hydrophilic.

In some embodiments, a subject microsphere comprises a first active agent and a second active agent, where the first active agent is associated with or linked to the nanoparticle or microparticle (e.g., the first active agent is present within the hydrophobic core, either free within the hydrophobic core, or Daltons, from about 50 Daltons to about 100 Daltons, from about 100 Daltons to about 500 Daltons, from about 500 Daltons to about 1 kDa, or from about 1 kDa to about 5 kDa).

Pharmacologically active agents useful for inclusion in a subject microsphere include drugs acting at synaptic and neuroeffector junctional sites (cholinergic agonists, anticholinesterase agents, atropine, scopolamine, and related antimuscarinic drugs, catecholamines and sympathomimetic drugs, and adrenergic receptor antagonists); drugs acting on the central nervous systems; autacoids (drug therapy of inflammation); drugs affecting renal function and electrolyte metabolism; cardiovascular drugs; drugs affecting gastrointestinal function; chemotherapy of neoplastic diseases; drugs acting on the blood and the blood-forming organs; and hormones and hormone antagonists. Thus, the agents useful in the composition include, but are not limited to anti-infectives such as antibiotics and antiviral agents; analgesics and analgesic combinations; local and general anesthetics; anorexics; antiarthritics; antiasthmtic agents; anticonvulsants; antidepressants; antihistamines; anti-inflammatory agents; antinauseants; antimigrane agents; antineoplastics; antipruritics; antipsychotics; antipyretics; antispasmodics; cardiovascular preparations (including calcium channel blockers, beta-blockers, beta-agonists and antiarrythmics); antihypertensives; diuretics; vasodilators; central nervous system stimulants; cough and cold preparations; decongestants; diagnostics; hormones; bone growth stimulants and bone resorptioninhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including double- and single-stranded molecules and supercoiled or condensed molecules, gene constructs, expression vectors, plasmids, antisense molecules and the like.

### Small Molecule Drugs

Any of a variety of small molecule active agents (“drugs”) can be included in a subject microsphere. Non-limiting examples include lipid-regulating agents; sex hormones; androgenic agents; antihypertensive agents; anti-diabetic agents; anti-viral agents; and active agents of any of the other below-listed categories.

Lipid-regulating agents that are generally classified as hydrophobic include HMG CoA reductase inhibitors such as atorvastatin, simvastatin, fluvastatin, pravastatin, lovastatin, cerivastatin, rosuvastatin, and pitavastatin, as well as other lipid-lowering (“antihyperlipidemic”) agents such as bezafibrate, beclobrate, binifibrate, ciprofibrate, clinofibrate, clofibrate, clofibric acid, ezetimibe, etofibrate, fenofibrate, fenofibric acid, gemfibrozil, nicofibrate, pirifibrate, probucol, ronifibrate, simfibrate, and theofibrate.

Sex hormones include, e.g., progestins (progestogens), estrogens, and combinations thereof. Progestins include acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, fluorogestone acetate, gestadene, hydroxyprogesterone, linked to a hydrophobic polymer in the hydrophobic core, or is linked to a hydrophilic polymer in the nanoparticle or microparticle); and the second agent is linked to or associated with the hydrogel. In some of these embodiments, the first active agent is hydrophobic and the second active agent is hydrophilic. Where a subject microsphere comprises a first active agent and a second active agent, where the first active agent is associated with or linked to the nanoparticle (or microparticle) and where the second active agent is associated with or linked to the hydrogel, a two-stage release profile is provided where the first active agent is released from the hydrogel at a first rate and over a first time period, and the second active agent is released from the nanoparticle (or microparticle) at a second rate and over a second time period.

In some embodiments, a first active agent present in the hydrogel is release from the hydrogel at a first rate, and a second active agent present in the nanoparticle (or microparticle) is release from the nanoparticle (or microparticle) at a second rate, where the first rate is higher (e.g., faster) than the second rate. In some embodiments, a first active agent present in the hydrogel is release from the hydrogel at a first rate, and a second active agent present in the nanoparticle (or microparticle) is release from the nanoparticle (or microparticle) at a second rate, where the first rate is lower (e.g., slower) than the second rate.

For example, in some embodiments, the first rate of release is such that from about 50% to about 100% (e.g., from about 50% to about 60%, from about 60% to about 70%, from about 70% to about 80%, from about 80% to about 90%, or from about 90% to about 100%) of the active agent is released over a period of time of from about 1 hour to about 48 hours (e.g., from about 1 hour to about 2 hours, from about 2 hours to about 4 hours, from about 4 hours to about 8 hours, from about 8 hours to about 12 hours, from about 12 hours to about 18 hours, from about 18 hours to about 24 hours, from about 24 hours to about 36 hours, or from about 36 hours to about 48 hours), or over a period of time of from about 2 days to about 12 days (e.g., from about 2 days to about 4 days, from about 4 days to about 6 days, from about 6 days to about 8 days, from about 8 days to about 10 days, or from about 10 days to about 12 days). As another example, in some embodiments, the second rate of release is such that from about 50% to about 100% (e.g., from about 50% to about 60%, from about 60% to about 70%, from about 70% to about 80%, from about 80% to about 90%, or from about 90% to about 100%) of the active agent is released over a period of time of from about 15 minutes to about 1 hour (e.g., from about 15 minutes to about 30 minutes, from about 30 minutes to about 45 minutes, or from about 45 minutes to about 60 minutes), or over a period of time of from about 1 hour to about 48 hours, (e.g., from about 1 hour to about 2 hours, from about 2 hours to about 4 hours, from about 4 hours to about 8 hours, from about 8 hours to about 12 hours, from about 12 hours to about 18 hours, from about 18 hours to about 24 hours, from about 24 hours to about 36 hours, or from about 36 hours to about 48 hours).

As another example, in some embodiments, the second rate of release is such that from about 50% to about 100% (e.g., from about 50% to about 60%, from about 60% to about 70%, from about 70% to about 80%, from about 80% to about 90%, or from about 90% to about 100%) of the active agent is released over a period of time of from about 1 hour to about 48 hours (e.g., from about 1 hour to about 2 hours, from about 2 hours to about 4 hours, from about 4 hours to about 8 hours, from about 8 hours to about 12 hours, from about 12 hours to about 18 hours, from about 18 hours to about 24 hours, from about 24 hours to about 36 hours, or from about 36 hours to about 48 hours), or over a period of time of from about 2 days to about 12 days (e.g., from about 2 days to about 4 days, from about 4 days to about 6 days, from about 6 days to about 8 days, from about 8 days to about 10 days, or from about 10 days to about 12 days). As another example, in some embodiments, the first rate of release is such that from about 50% to about 100% (e.g., from about 50% to about 60%, from about 60% to about 70%, from about 70% to about 80%, from about 80% to about 90%, or from about 90% to about 100%) of the active agent is released over a period of time of from about 15 minutes to about 1 hour (e.g., from about 15 minutes to about 30 minutes, from about 30 minutes to about 45 minutes, or from about 45 minutes to about 60 minutes), or over a period of time of from about 1 hour to about 48 hours, (e.g., from about 1 hour to about 2 hours, from about 2 hours to about 4 hours, from about 4 hours to about 8 hours, from about 8 hours to about 12 hours, from about 12 hours to about 18 hours, from about 18 hours to about 24 hours, from about 24 hours to about 36 hours, or from about 36 hours to about 48 hours).

### Nanoparticle and Microparticle Polymers

The nanoparticle or microparticle can have an average diameter of from about 1 nm to about 900 μm, e.g., the nanoparticle can have an average diameter of from about 1 nm to about 5 nm, from about 5 nm to about 25 nm, from about 25 nm to about 50 nm, from about 50 nm to about 75 nm, from about 75 nm to about 100 nm, from about 100 nm to about 200 nm, from about 200 nm to about 300 nm, from about 300 nm to about 400 nm, from about 400 nm to about 500 nm, from about 500 nm to about 600 nm, from about 600 nm to about 700 nm, from about 700 nm to about 800 nm, from about 800 nm to about 900 nm, from about 900 nm to about 1 μm, from about 1 μm to about 10 μm, from about 10 μm to about 25 μm, from about 25 μm to about 50 μm, from about 50 μm to about 75 μm, from about 75 μm to about 100 μm, from about 100 μm to about 200 μm, from about 200 μm to about 300 μm, from about 300 μm to about 400 μm, from about 400 μm to about 500 μm, from about 500 μm to about 600 μm, from about 600 μm to about 700 μm, from about 700 μm to about 800 μm, or from about 800 μm to about 900 μm.

The nanoparticle or microparticle comprises a hydrophobic polymer and a hydrophilic polymer. Suitable hydrophobic and hydrophilic polymers include biocompatible polymers comprising from about 50 to about 100,000 subunits, e.g., from about 50 subunits to about 100 subunits, from about 100 subunits to about 500 subunits, from about 500 subunits to about 1,000 subunits, from about 1,000 subunits to about 5,000 subunits, from about 5,000 subunits to about 10,000 subunits, from about 10,000 subunits to about 25,000 subunits, from about 25,000 subunits to about 50,000 subunits, or from about 50,000 subunits to about 100,000 subunits. In some embodiments, the linear polymer comprises more than 100,000 subunits.

The subunits can all be identical, e.g., the polymer is a homopolymer. In other embodiments, more than one species of subunit is present, e.g., the polymer is a heteropolymer or co-polymer. In some embodiments, the polymer is a linear polymer. In other embodiments, the polymer may include one or more branches.

Suitable polymers include natural polymers, semisynthetic polymers, and synthetic polymers. Suitable synthetic polymers include, but are not limited to, polymers or copolymers derived from polydioxane, polyphosphazene, polysulphone resins, poly(acrylic acid), poly(acrylic acid) butyl ester, poly(ethylene glycol), poly(propylene), polyurethane resins, poly(methacrylic acid), poly(methacrylic acid)-methyl ester, poly(methacrylic acid)-n butyl ester, poly(methacrylic acid)-t butyl ester, polytetrafluoroethylene, polyperfluoropropylene, poly N-vinyl carbazole, poly(methyl isopropenyl ketone), poly alphamethyl styrene, polyvinylacetate, poly(oxymethylene), poly(ethylene-co-vinyl acetate), a polyurethane, a poly(vinyl alcohol), and polyethylene terephthalate; ethylene vinyl alcohol copolymer (commonly known by the generic name EVOH or by the trade name EVAL); polybutylmethacrylate; poly(hydroxyvalerate); poly(L-lactic acid) or poly(L-lactide); poly(e-caprolactone); poly(lactide-co-glycolide); poly(hydroxybutyrate); poly(hydroxybutyrate-co-valerate); polydioxanone; polyorthoester; polyanhydride; poly(glycolic acid) (PGA); poly(D,L-lactide) (PDLL); poly(L-Lactide)(PLL); copolymers of PGA, poly(D,L-lactic acid) (PDLA), and/or poly(lactic acid) (PLA); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxid) (PEO)/PLA); polyalkylene oxalates; polyphosphazenes; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; and carboxymethyl cellulose.

Suitable hydrophobic polymers include poly(L-lactide), poly(glycolide), poly(e-caprolactone), copolymers of lactide and/or glycolide or/and poly(e-caprolactone), hydrophobic peptides or a combination of hydrophobic peptides, polyurethanes. Any hydrophobic polymer that can form a micelle in water is suitable for use as a hydrophobic polymer. Suitable hydrophobic polymers include, e.g., poly(glycolide) or poly(glycolic acid); poly(e-caprolactone); poly(D,L-lactide); poly (L-Lactide); copolymers of these and other polyesters; polyamides; polyanhydrides; polyurethanes; poly(ortho esters); poly(iminocarbonates). In some embodiments, the hydrophobic polymer of the nanoparticle (or microparticle) is poly-L-lactide.

Suitable hydrophilic polymers include, but are not limited to, poly(ethylene glycol); poly(vinyl alcohol); polyethers; poly(methacrylic acid); poly(acrylic acid); poly(hydroxyethylmethacrylate) (pHEMA); hyaluronic acid; and hyaluronate.

In some embodiments, the hydrophilic polymer of the nanoparticle or microparticle is a poly(ethylene glycol) polymer. Polyethylene glycol has the general formula R(O—CH₂—CH₂)ₙO—R, where R is hydrogen or a protective group such as an alkyl or an alkanol group, and where n is an integer from 1 to 1000.

Poly(ethylene glycol) (PEG) having a molecular weight in a range of from about 2 kDa to about 100 kDa, can be used, where the term “about,” in the context of PEG, indicates that in preparations of polyethylene glycol, some molecules will weigh more, some less, than the stated molecular weight. For example, PEG suitable for conjugation to IFN-α has a molecular weight of from about 2 kDa to about 5 kDa, from about 5 kDa to about 10 kDa, from about 10 kDa to about 15 kDa, from about 15 kDa to about 20 kDa, from about 20 kDa to about 25 kDa, from about 25 kDa to about 30 kDa, from about 30 kDa to about 40 kDa, from about 40 kDa to about 50 kDa, from about 50 kDa to about 60 kDa, from about 60 kDa to about 70 kDa, from about 70 kDa to about 80 kDa, from about 80 kDa to about 90 kDa, or from about 90 kDa to about 100 kDa.

In some embodiments, the PEG is linear. In other embodiments, the PEG is branched. Branched PEG derivatives such as those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

PEG has at least one hydroxyl group, e.g., a terminal hydroxyl group, which hydroxyl group can be modified to generate a functional group that is reactive with an amino group, e.g., an epsilon amino group of a lysine residue, a free amino group at the N-terminus of a polypeptide, or any other amino group such as an amino group of asparagine, glutamine, arginine, or histidine.

The PEG can be derivatized so that an active agent can be linked to the PEG polymer. Suitable derivatives of PEG that are reactive with the free carboxyl group at the carboxyl-terminus of peptide include, but are not limited to PEG-amine, and hydrazine derivatives of PEG (e.g., PEG-NH—NH₂). The PEG can be methoxy-PEG, e.g., monomethoxy-PEG.

A PEG polymer can be derivatized such that it comprises a terminal thiocarboxylic acid group, —COSH, which selectively reacts with amino groups to generate amide derivatives. Because of the reactive nature of the thio acid, selectivity of certain amino groups over others is achieved. For example, —SH exhibits sufficient leaving group ability in reaction with N-terminal amino group at appropriate pH conditions such that the E-amino groups in lysine residues are protonated and remain non-nucleophilic. On the other hand, reactions under suitable pH conditions may make some of the accessible lysine residues to react with selectivity.

PEG can comprise a reactive ester such as an N-hydroxy succinimidate at the end of the PEG chain. Such an N-hydroxysuccinimidate-containing PEG molecule reacts with select amino groups at particular pH conditions such as neutral 6.5-7.5. For example, the N-terminal amino groups may be selectively modified under neutral pH conditions. However, if the reactivity of the reagent were extreme, accessible —NH₂ groups of lysine may also react.

An active agent can be coupled directly to PEG (i.e., without a linking group) through an amino group, a sulfhydryl group, a hydroxyl group, or a carboxyl group.

An active agent can be attached to the PEG via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl butanoate (SBA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine.

A non-limiting example of a suitable co-polymer forming a nanoparticle or microparticle is a poly(lysine-g(lactide-b-ethylene glycol) terpolymer. Park and Healy (2003) Bioconjugate Chem 14: 31119.

### Hydrogel

Suitable hydrogel monomers include the following: lactic acid, glycolic acid, acrylic acid, 1-hydroxyethyl methacrylate (HEMA), ethyl methacrylate (EMA), propylene glycol methacrylate (PEMA), acrylamide (AAM), N-vinylpyrrolidone, methyl methacrylate (MMA), glycidyl methacrylate (GDMA), glycol methacrylate (GMA), ethylene glycol, fumaric acid, and the like. Common cross linking agents include tetraethylene glycol dimethacrylate (TEGDMA) and N,N′-methylenebisacrylamide. The hydrogel can be homopolymeric, or can comprise co-polymers of two or more of the aforementioned polymers.

The hydrogel that surrounds the nanoparticle or microparticle is generally hydrophilic. Suitable polymers for inclusion in the hydrogel include, but are not limited to, poly(N-isopropylacrylamide) (pNIPAAm); poly(N-isopropylacrylamide-co-acrylic acid); hyaluronic acid or hyaluronate; crosslinked hyaluronic acid or hyaluronate; pHEMA; or copolymers of p(NIPAAm)-based sIPNs and other hydrogel sIPNs (semi-interpenetrating networks).

In some embodiments, the hydrogel is a temperature-sensitive hydrogel. In some embodiments, a temperature-sensitive hydrogel is a polyacrylic acid or derivative thereof, e.g., poly (N-isopropylacrylamide) gel, and the increase in temperature causes the hydrogel to contract, thereby forcing the active agent out of the hydrogel. Alternatively, the temperature-sensitive hydrogel is an interpenetrating hydrogel network of poly(acrylamide) and poly(acrylic acid), and the increase in temperature causes the hydrogel to swell, thereby allowing the active agent to diffuse out of the gel. The temperature required for triggering release of an active agent from the hydrogel is generally about normal body temperature, e.g., about 37° C.

One or more of the hydrogel polymers can be modified with a cell-binding moiety, e.g., a moiety that provides for binding to a cell-surface receptor. For example, a cell-binding moiety can include an Arg-Gly-Asp (RGD) peptide. A suitable RGD peptide comprises the amino acid sequence: CGGNGEPRGDTYRAY (SEQ ID NO:1). Also suitable for use are peptides comprising the amino acid sequence FHRRIKA (SEQ ID NO:2). Also suitable for use are the peptides acetyl-CGGNGEPRGDTYRAY-NH₂ (SEQ ID NO:3) and acetyl-CGGFHRRIKA-NH₂ (SEQ ID NO:4). Other suitable peptides are shown in Table 2, below.

TABLE 2

| Peptide | SEQ ID NO: |
|---|---|
| CGGNGEPRGDTYRAY | SEQ ID NO: 1 |
| C*EPRGDTYRAYG* | SEQ ID NO: 5 |
| CGGGEAPRGDVY | SEQ ID NO: 6 |
| C*CGPRGDVYG* | SEQ ID NO: 7 |
| CGGVSWFSRHRYSPFAVS | SEQ ID NO: 8 |
| CGGNRWHSIYITRFG | SEQ ID NO: 9 |
| CGGTWYKIAFQRNRK | SEQ ID NO: 10 |
| CGGRKRLQVQLSIRT | SEQ ID NO: 11 |
| CGGKAFDITYVRLKF | SEQ ID NO: 12 |
| C*TRKKHDNAQ* | SEQ ID NO: 13 |
| VSWFSRHRYSPFAVS | SEQ ID NO: 14 |
| RNIAEIIKDI | SEQ ID NO: 15 |
| TAGSCLRKFSTM | SEQ ID NO: 16 |
| TTSWSQCSKS | SEQ ID NO: 17 |
| RYVVLPRPVCFEK | SEQ ID NO: 18 |
| EVLLI | SEQ ID NO: 19 |

As noted above, in some embodiments, the hydrogel comprises an active agent linked to one or more moieties in the hydrogel, or embedded in the hydrogel. In some embodiments, the active agent is a hydrophilic compound.

The hydrogel can be modified with one or more proteolytically cleavable crosslinks. See, e.g., Kim and Healy (2003) Biomacromolecules 4:1214. For example, the proteolytically cleavable crosslink can be a matrix metalloproteinase cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue), e.g., Pro-X-X-Hy-(Ser/Thr), e.g., Pro-Leu/Gln-Gly-Met-Thr-Ser (SEQ ID NO:20) or Pro-Leu/Gln-Gly-Met-Thr (SEQ ID NO:21). Another example of a protease cleavage site is a plasminogen activator cleavage site, e.g., a uPA or a tissue plasminogen activator (tPA) cleavage site. Specific examples of cleavage sequences of uPA and tPA include sequences comprising Val-Gly-Arg. Another example is a thrombin cleavage site, e.g., CGLVPAGSGP (SEQ ID NO:22). Additional suitable linkers comprising protease cleavage sites include linkers comprising one or more of the following amino acid sequences: 1) SLLKSRMVPNFN (SEQ ID NO:23) or SLLIARRMPNFN (SEQ ID NO:24), cleaved by cathepsin B; SKLVQASASGVN (SEQ ID NO:25) or SSYLKASDAPDN (SEQ ID NO:26), cleaved by an Epstein-Barr virus protease; RPKPQQFFGLMN (SEQ ID NO:27) cleaved by MMP-3 (stromelysin); SLRPLALWRSFN (SEQ ID NO:28) cleaved by MMP-7 (matrilysin); SPQGIAGQRNFN (SEQ ID NO:29) cleaved by MMP-9; DVDERDVRGFASFL SEQ ID NO:30) cleaved by a thermolysin-like MMP; SLPLGLWAPNFN (SEQ ID NO:31) cleaved by matrix metalloproteinase 2(MMP-2); SLLIFRSWANFN (SEQ ID NO:32) cleaved by cathespin L; SGVVIATVIVIT (SEQ ID NO:33) cleaved by cathespin D; SLGPQGIWGQFN cleaved by matrix metalloproteinase 1(MMP-1); KKSPGRVVGGSV (SEQ ID NO:34) cleaved by urokinase-type plasminogen activator; PQGLLGAPGILG (SEQ ID NO:35) cleaved by membrane type 1 matrixmetalloproteinase (MT-MMP); HGPEGLRVGFYESDVMGRGHARLVHVEEPHT (SEQ ID NO:36) cleaved by stromelysin 3 (or MMP-11), thermolysin, fibroblast collagenase and stromelysin-1; GPQGLAGQRGIV (SEQ ID NO:37) cleaved by matrix metalloproteinase 13 (collagenase-3); GGSGQRGRKALE (SEQ ID NO:38) cleaved by tissue-type plasminogen activator(tPA); SLSALLSSDIFN (SEQ ID NO:39) cleaved by human prostate-specific antigen; SLPRFKIIGGFN (SEQ ID NO:40) cleaved by kallikrein (hK3); SLLGIAVPGNFN (SEQ ID NO:41) cleaved by neutrophil elastase; and FFKNIVTPRTPP (SEQ ID NO:42) cleaved by calpain (calcium activated neutral protease).

### Active Agents

Active agents that can be included in a subject biodegradable microsphere include, but are not limited to, small molecule drugs, peptides, microRNAs (miRNA), and interfering RNAs. Small molecule drugs include drugs having a molecular weight of from about 5 Daltons to about 50 kDaltons (kDa) (e.g., from about 5 Daltons to about 10 Daltons, from about 10 Daltons to about 50 Daltons, from about 50 Daltons to about 100 Daltons, from about 100 Daltons to about 500 Daltons, from about 500 Daltons to about 1 kDa, from about 1 kDa to about 5 kDa, from about 5 kDa to about 10 kDa, from about 10 kDa to about 25 kDa, or from about 25 kDa to about 50 kDa), or from about 5 Daltons to about 5 kDa (e.g., from about 5 Daltons to about 10 Daltons, from about 10 Daltons to about 50 hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, progesterone, and trimgestone. Also included within this general class are estrogens, e.g.: estradiol (i.e., 1,3,5-estratriene-3,17β-diol, or “17β-estradiol”) and its esters, including estradiol benzoate, valerate, cypionate, heptanoate, decanoate, acetate and diacetate; 2-Methoxyestradiol; 4-Hydroxyestradiol; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. In many contexts, e.g., in female contraception and in hormone replacement therapy (HRT), a combination of a progestin and estrogen is used, e.g., progesterone and 17β-estradiol. For HRT, an androgenic agent may be advantageously included as well. Androgenic agents for this purpose include, for example, dehydroepiandrosterone (DHEA; also termed “prasterone”), sodium dehydroepiandrosterone sulfate, 4-dihydrotestosterone (DHT; also termed “stanolone”), and testosterone, and pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters.

Other androgenic agents include, but are not limited to, androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, and dromostanolone propionate.

Antihypertensive agents include, without limitation, amlodipine, benazepril, benidipine, candesartan, captopril, carvedilol, darodipine, dilitazem, diazoxide, doxazosin, enalapril, epleronone, eposartan, felodipine, fenoldopam, fosinopril, guanabenz, iloprost, irbesartan, isradipine, lercardinipine, lisinopril, losartan, minoxidil, nebivolol, nicardipine, nifedipine, nimodipine, nisoldipine, omapatrilat, phenoxybenzamine, prazosin, quinapril, reserpine, semotiadil, sitaxsentan, terazosin, telmisartan, and valsartan.

Anti-diabetic agents include, by way of example, acetohexamide, chlorpropamide, ciglitazone, farglitazar, glibenclamide, gliclazide, glipizide, glucagon, glyburide, glymepiride, miglitol, pioglitazone, nateglinide, pimagedine, repaglinide, rosiglitazone, tolazamide, tolbutamide, triampterine, and troglitazone.

Antiviral agents that can be delivered using the present methods and dosage forms include the antiherpes agents acyclovir, famciclovir, foscarnet, ganciclovir, idoxuridine, sorivudine, trifluridine, valacyclovir, and vidarabine, and otherantiviral agents such as abacavir, amantadine, amprenavir, delviridine, didanosine, efavirenz, indinavir, interferon alpha, lamivudine, nelfinavir, nevirapine, ribavirin, rimantadine, ritonavir, saquinavir, stavudine, tipranavir, valganciclovir, zalcitabine, and zidovudine; and other antiviral agents such as abacavir, indinavir, interferon alpha, nelfinavir, ribavirin, rimantadine, tipranavir, ursodeoxycholic acid, and valganciclovir.

Additional suitable active agents include:

anti-inflammatory agents and non-opioid analgesics, such as aloxiprin, auranofin, azapropazone, azathioprine, benorylate, butorphenol, capsaicin, celecoxib, diclofenac, diflunisal, esonarimod, etodolac, fenbufen, fenoprofen calcium, flurbiprofen, ibuprofen, indomethacin, ketoprofen, ketorolac, leflunomide, meclofenamic acid, mefenamic acid, nabumetone, naproxen, novantrone, oxaprozin, oxyphenbutazone, parecoxib, phenylbutazone, piclamilast, piroxicam, rofecoxib, ropivacaine, sulindac, tetrahydrocannabinol, tramadol, tromethamine, valdecoxib, and ziconotide, as well as the urinary analgesics phenazopyridine and tolterodine;

anti-angina agents, such as mibefradil, refludan, nahnefene, carvedilol, cromafiban, lamifiban, fasudil, ranolazine, tedisamil, nisoldipine, and tizanidine;

antihelminthics, such as albendazole, bephenium hydroxynaphtho ate, cambendazole, dichlorophen, ivermectin, mebendazole, oxamniquine, oxfendazole, oxantel embonate, praziquantel, pyrantel embonate and thiabendazole;

anti-arrhythmic agents, such as amiodarone, disopyramide, flecamide acetate and quinidine sulfate;

anti-asthma agents, such as zileuton, zafirlukast, terbutaline sulfate, montelukast, and albuterol;

anti-bacterial agents, such as alatrofloxacin, azithromycin, baclofen, benethamine penicillin, cinoxacin, ciprofloxacin, clarithromycin, clofazimine, cloxacillin, demeclocycline, dirithromycin, doxycycline, erythromycin, ethionamide, furazolidone, grepafloxacin, imipenem, levofloxacin, lorefloxacin, moxifloxacin, nalidixic acid, nitrofurantoin, norfloxacin, ofloxacin, rifampicin, rifabutine, rifapentine, sparfloxacin, spiramycin, sulphabenzamide, sulphadoxine, sulphamerazine, sulphacetamide, sulphadiazine, sulphafurazole, sulphamethoxazole, sulphapyridine, tetracycline, trimethoprim, trovafloxacin, and vancomycin;

anti-cancer agents and immunosuppressants, such as alitretinoin, aminoglutethimide, amsacrine, anastrozole, azathioprine, bexarotene, bicalutamide, biricodar, bisantrene, busulfan, camptothecin, candoxatril, capecitabine, cytarabine, chlorambucil, cyclosporin, dacarbazine, decitabine, ellipticine, estramustine, etoposide, gemcitabine, irinotecan, lasofoxifene, letrozole, lomustine, melphalan, mercaptopurine, methotrexate, mitomycin, mitotane, mitoxantrone, mofetil, mycophenolate, nebivolol, nilutamide, paclitaxel, palonosetron, procarbazine, ramipril, rubitecan, sirolimus, tacrolimus, tamoxifen, teniposide, testolactone, thalidomide, tirapazamine, topotecan, toremifene citrate, vitamin A, vitamin A derivatives, and zacopride;

anti-coagulants and other agents for preventing and treating stroke, such as cilostazol, citicoline, clopidogrel, cromafiban, dexanabinol, dicumarol, dipyridamole, nicoumalone, oprelvekin, perindopril erbumine, phenindione, ramipril, repinotan, ticlopidine, tirofiban, and heparin, including heparin salts formed with organic or inorganic bases, and low molecular weight heparin, i.e., heparin fragments generally having a weight average molecular weight in the range of about 1000 to about 10,000 D and exemplified by enoxaparin, dalteparin, danaproid, gammaparin, nadroparin, ardeparin, tinzaparin, certoparin, and reviparin;

anti-diabetics, such as acetohexamide, chlorpropamide, farglitazar, glibenclamide, gliclazide, glipizide, glimepiride, miglitol, nateglinide, pimagedine, pioglitazone, repaglinide, rosiglitazone, tolazamide, tolbutamide, troglitazone, and voglibose;

anti-epileptics, such as beclamide, carbamazepine, clonazepam, ethotoin, felbamate, fosphenyloin, lamotrigine, methoin, methsuximide, methylphenobarbitone, oxcarbazepine, paramethadione, phenacemide, phenobarbitone, phenyloin, phensuximide, primidone, sulthiame, tiagabine, topiramate, valproic acid, and vigabatrin;

anti-fungal agents, such as amphotericin, butenafine, butoconazole nitrate, clotrimazole, econazole nitrate, fluconazole, flucytosine, griseofulvin, itraconazole, ketoconazole, miconazole, natamycin, nystatin, sulconazole nitrate, oxiconazole, terbinafine, terconazole, tioconazole and undecenoic acid;

anti-gout agents, such as allopurinol, probenecid and sulphin-pyrazone;

antihistamines and allergy medications, such as acrivastine, astemizole, chlorpheniramine, cinnarizine, cetirizine, clemastine, cyclizine, cyproheptadine, desloratadine, dexchlorpheniramine, dimenhydrinate, diphenhydramine, epinastine, fexofenadine, flunarizine, loratadine, meclizine, mizolastine, oxatomide, and terfenadine;

anti-malarials, such as amodiaquine, chloroquine, chlorproguanil, halofantrine, mefloquine, proguanil, pyrimethamine and quinine sulfate;

agents for treating headaches, including anti-migraine agents, such as almotriptan, butorphanol, dihydroergotamine, dihydroergotamine mesylate, eletriptan, ergotamine, frovatriptan, methysergide, naratriptan, pizotyline, rizatriptan, sumatriptan, tonaberstat, and zolmitriptan;

anti-muscarinic agents, such as atropine, benzhexyl, biperiden, ethopropazine, hyoscyamine, mepenzolate bromide, oxyphencyclimine, scopolamine, and tropicamide;

anti-protozoal agents, such as atovaquone, benznidazole, clioquinol, decoquinate, diiodohydroxyquinoline, diloxanide furoate, dinitolmide, furazolidone, metronidazole, nimorazole, nitrofirazone, ornidazole and tinidazole;

anti-thyroid agents, such as carbimazole, paricalcitol, and propylthiouracil;

anti-tussives, such as benzonatate;

anxiolytics, sedatives, and hypnotics, such as alprazolam, amylobarbitone, barbitone, bentazepam, bromazep am, bromperidol, brotizolam, butobarbitone, carbromal, chlordiazepoxide, chlormethiazole, chlorpromazine, chlorprothixene, clonazepam, clobazam, clotiazepam, clozapine, dexmethylphenidate (d-threo-methylphenidate) diazepam, droperidol, ethinamate, flunanisone, flunitrazepam, triflupromazine, flupenthixol decanoate, fluphenazine, flurazepam, gabapentin, gaboxadol, γ-hydroxybutyrate, haloperidol, lamotrigine, lorazepam, lormetazepam, medazepam, meprobamate, mesoridazine, methaqualone, methylphenidate, midazolam, modafinil, molindone, nitrazepam, olanzapine, oxazepam, pentobarbitone, perphenazine pimozide, pregabalin, prochlorperazine, pseudoephedrine, quetiapine, rispiridone, sertindole, siramesine, sulpiride, sunepitron, temazepam, thioridazine, triazolam, zaleplon, zolpidem, and zopiclone;

appetite suppressants, anti-obesity drugs and drugs for treatment of eating disorders, such as amphetamine, bromocriptine, dextroamphetamine, diethylpropion, lintitript, mazindol, methamphetamine, orlistat, phentermine, and topiramate;

cardiovascular drugs, including: angiotensin converting enzyme (ACE) inhibitors such as enalapril, ramipril, perindopril erbumine, 1-carboxymethyl-3-1-carboxy-3-phenyl-(1S)-propylamino-2,3,4,5-tetrahydro-1H-(3S)-1-benzazepine-2-one, 3-(5-amino-1-carboxy-1S-pentyl)amino-2,3,4,5-tetrahydro-2-oxo-3S-1H-1-ben-zazepine-lacetic acid or 3-(1-ethoxycarbonyl-3-phenyl-(1S)-propylamino)-2,3,4,5-tetrahydro-2-oxo-(−3S)-benzazepi acid monohydrochloride; cardiac glycosides and cardiac inotropes such as aminone, digoxin, digitoxin, enoximone, lanatoside C, medigoxin, and milrinone; calcium channel blockers such as verapamil, nifedipine, nicardipene, felodipine, isradipine, nimodipine, amlodipine and diltiazem; beta-blockers such as acebutolol, alprenolol, atenolol, labetalol, metoprolol, nadolol, oxyprenolol, pindolol, propafenone, propranolol, esmolol, sotalol, timolol, and acebutolol; antiarrhythmics such as moricizine, dofetilide, ibutilide, nesiritide, procainamide, quinidine, disopyramide, lidocaine, phenyloin, tocamide, mexiletine, flecamide, encamide, bretylium and amiodarone; cardioprotective agents such as dexrazoxane and leucovorin; vasodilators such as nitroglycerin; diuretic agents such as azetazolamide, amiloride, bendroflumethiazide, bumetanide, chlorothiazide, chlorthalidone, ethacrynic acid, furosemide, hydrochlorothiazide, metolazone, nesiritide, spironolactone, and triamterine; and miscellaneous cardiovascular drugs such as monteplase and corlopam;

corticosteroids, such as beclomethasone, betamethasone, budesonide, cortisone, desoxymethasone, dexamethasone, fludrocortisone, flunisolide, fluocortolone, fluticasone propionate, hydrocortisone, methylprednisolone, prednisolone, prednisone and triamcinolone;

erectile dysfunction drugs, such as apomorphine, phentolamine, and vardenafil;

gastrointestinal agents, such as alosetron, bisacodyl, cilansetron, cimetidine, cisapride, diphenoxylate, domperidone, esomeprazole, famotidine, granisetron, lansoprazole, loperamide, mesalazine, nizatidine, omeprazole, ondansetron, prantoprazole, rabeprazole sodium, ranitidine, risperidone, sulphasalazine, and tegaserod;

keratolytics, such as such as acetretin, calcipotriene, calcifediol, calcitriol, cholecalciferol, ergocalciferol, etretinate, retinoids, targretin, and tazarotene;

lipid regulating agents, such as atorvastatin, bezafibrate, cerivastatin, ciprofibrate, clofibrate, ezetimibe, fenofibrate, fluvastatin, gemfibrozil, pitavastatin, pravastatin, probucol, rosuvastatin, and simvastatin;

muscle relaxants, such as cyclobenzaprine, dantrolene sodium and tizanidine HCl;

agents to treat neurodegenerative diseases, including active agents for treating Alzheimer's disease such as akatinol, donezepil, donepezil hydrochloride, dronabinol, galantamine, neotrofin, rasagiline, physostigmine, physostigmine salicylate, propentoffyline, quetiapine, rivastigmine, tacrine, tacrine hydrochloride, thalidomide, and xaliproden; active agents for treating Huntington's Disease, such as fluoxetine and carbamazepine; anti-parkinsonism drugs useful herein include amantadine, apomorphine, bromocriptine, entacapone, levodopa (particularly a levodopa/carbidopa combination), lysuride, pergolide, pramipexole, rasagiline, riluzole, ropinirole, selegiline, sumanirole, tolcapone, trihexyphenidyl, and trihexyphenidyl hydrochloride; and active agents for treating ALS such as the anti-spastic agents baclofen, diazemine, and tizanidine;

nitrates and other anti-anginal agents, such as amyl nitrate, glyceryl trinitrate, isosorbide dinitrate, isosorbide mononitrate and pentaerythritol tetranitrate;

neuroleptic drugs, including antidepressant drugs, antimanic drugs, and antipsychotic agents, wherein antidepressant drugs include (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors citalopram, fluoxetine, fluvoxamine, paroxetine, sertraline, and venlafaxine, (c) monoamine oxidase inhibitors such as phenelzine, tranylcypromine, and (−)-selegiline, and (d) other antidepressants such as aprepitant, bupropion, duloxetine, gepirone, igmesine, lamotrigine, maprotiline, mianserin, mirtazapine, nefazodone, rabalzotan, sunepitron, trazodone and venlafaxine, and wherein antimanic and antipsychotic agents include (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, pimozide, quetiapine, risperidone, and sertindole;

nutritional agents, such as calcitriol, carotenes, dihydrotachysterol, essential fatty acids, non-essential fatty acids, phytonadiol, vitamin A, vitamin B₂, vitamin D, vitamin E and vitamin K;

opioid analgesics, such as alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, and tramadol; and

stimulants, including active agents for treating narcolepsy, attention deficit disorder (ADD) and attention deficit hyperactivity disorder (ADHD), such as amphetamine, dexamphetamine, dexfenfluramine, fenfluramine, mazindol, methylphenidate (including d-threo-methylphenidate, or “dexmethylphenidate,” as well as racemic d,l-threo-methylphenidate), modafinil, pemoline, and sibutramine.

### Hydrophobic Active Agents

Non-limiting examples of hydrophobic active agents include, but are not limited to, acetretin, acetyl coenzyme Q, albendazole, albuterol, aminoglutethimide, amiodarone, amlodipine, amphetamine, amphotericin B, atorvastatin, atovaquone, azithromycin, baclofen, beclomethasone, benazepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicumarol, digoxin, dehydroepiandrosterone, dihydroergotamine, dihydrotachysterol, dirithromycin, donezepil, efavirenz, eposartan, ergocalciferol, ergotamine, essential fatty acid sources, estradiol, etodolac, etoposide, famotidine, fenofibrate, fentanyl, fexofenadine, finasteride, fluconazole, flurbiprofen, fluvastatin, fosphenyloin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thyroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nisoldipine, nilutanide, nitro furantoin, nizatidine, omeprazole, oprevelkin, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, repaglinide, rifabutine, rifapentine, rimexolone, ritanovir, rizatriptan, rofecoxib, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofiban, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, zopiclone, and combinations thereof.

### Hydrophilic Active Agents

Non-limiting examples of hydrophilic active agents include, without limitation, acarbose, acyclovir, acetyl cysteine, acetylcholine chloride, alatrofloxacin, alendronate, alglucerase, amantadine hydrochloride, ambenomium, amifostine, amiloride hydrochloride, aminocaproic acid, amphotericin B, antihemophilic factor (human), antihemophilic factor (porcine), antihemophilic factor (recombinant), aprotinin, asparaginase, atenolol, atracurium besylate, atropine, azithromycin, aztreonam, BCG vaccine, bacitracin, becaplermin, belladona, bepridil hydrochloride, bleomycin sulfate, calcitonin human, calcitonin salmon, carboplatin, capecitabine, capreomycin sulfate, cefamandole nafate, cefazolin sodium, cefepime hydrochloride, cefixime, cefonicid sodium, cefoperazone, cefotetan disodium, cefotaxime, cefoxitin sodium, ceftizoxime, ceftriaxone, cefuroxime axetil, cephalexin, cephapirin sodium, cholera vaccine, chorionic gonadotropin, cidofovir, cisplatin, cladribine, clidinium bromide, clindamycin and clindamycin derivatives, ciprofloxacin, clodronate, colistimethate sodium, colistin sulfate, corticotropin, cosyntropin, cromolyn sodium, cytarabine, dalteparin sodium, danaparoid, deferoxamine, denileukin diftitox, desmopressin, diatrizoate meglumine and diatrizoate sodium, dicyclomine, didanosine, dirithromycin, dopamine hydrochloride, dornase alpha, doxacurium chloride, doxorubicin, etidronate disodium, enalaprilat, enkephalin, enoxaparin, enoxaprin sodium, ephedrine, epinephrine, epoetin alpha, erythromycin, esmolol hydrochloride, factor IX, famciclovir, fludarabine, fluoxetine, foscamet sodium, ganciclovir, granulocyte colony stimulating factor, granulocyte-macrophage stimulating factor, recombinant human growth hormone, bovine growth hormone, gentamycin, glucagon, glycopyrolate, gonadotropin releasing hormone and synthetic analogs thereof, gonadorelin, grepafloxacin, haemophilus B conjugate vaccine, hepatitis A virus vaccine inactivated, hepatitis B virus vaccine inactivated, heparin sodium, indinavir sulfate, influenza virus vaccine, interleukin-2, interleukin-3, insulin-human, insulin lispro, insulin procine, insulin NPH, insulin aspart, insulin glargine, insulin detemir, interferon alpha, interferon beta, ipratropium bromide, ifosfamide, Japanese encephalitis virus vaccine, lamivudine, leucovorin calcium, leuprolide acetate, levofloxacin, lincomycin and lincomycin derivatives, lobucavir, lomefloxacin, loracarbef, mannitol, measles virus vaccine, meningococcal vaccine, menotropins, mepenzolate bromide, mesalamine, methenamine, methotrexate, methscopolamine, metformin hydrochloride, metoprolol, mezlocillin sodium, mivacurium chloride, mumps viral vaccine, nedocromil sodium, neostigmine bromide, neostigmine methyl sulfate, neurontin, norfloxacin, octreotide acetate, ofloxacin, olpadronate, oxytocin, pamidronate disodium, pancuronium bromide, paroxetine, perfloxacin, pentamidine isethionate, pentostatin, pentoxifylline, penciclovir, pentagastrin, phentolamine mesylate, phenylalanine, physostigmine salicylate, plague vaccine, piperacillin sodium, platelet derived growth factor, pneumococcal vaccine polyvalent, poliovirus vaccine (inactivated), poliovirus vaccine live (OPV), polymyxin B sulfate, pralidoxime chloride, pramlintide, pregabalin, propafenone, propenthaline bromide, pyridostigmine bromide, rabies vaccine, risedronate, ribavirin, rimantadine hydrochloride, rotavirus vaccine, salmeterol xinafoate, sincalide, small pox vaccine, solatol, somatostatin, sparfloxacin, spectinomycin, stavudine, streptokinase, streptozocin, suxamethonium chloride, tacrine hydrochloride, terbutaline sulfate, thiopeta, ticarcillin, tiludronate, timolol, tissue type plasminogen activator, TNFR:Fc, TNK-tPA, trandolapril, trimetrexate gluconate, trospectomycin, trovafloxacin, tubocurarine chloride, tumor necrosis factor, typhoid vaccine live, urea, urokinase, vancomycin, valacyclovir, valsartan, varicella virus vaccine live, vasopressin and vasopressin derivatives, vecuronium bromide, vinblastine, vincristine, vinorelbine, vitamin B12, warfarin sodium, yellow fever vaccine, zalcitabine, zanamivir, zolendronate, zidovudine, and combinations thereof.

### Polypeptide Agents

Peptidyl drugs include therapeutic peptides and proteins per se, whether naturally occurring, chemically synthesized, recombinantly produced, and/or produced by biochemical (e.g., enzymatic) fragmentation of larger molecules, and may contain the native sequence or an active fragment thereof. Specific peptidyl drugs include, without limitation, the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP), and vasopressin. Other peptidyl drugs are the cytokines, e.g., colony stimulating factor 4, heparin binding neurotrophic factor (HBNF), interferon-α, interferon α-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage colony-stimulating factor (GM-CSF), macrophage colony-stimulating factor, midkine (MD), and thymopoietin. Still other peptidyl drugs that can be advantageously delivered using the methodology and formulations of the present invention include endorphins (e.g., dermorphin, dynorphin, α-endorphin, β-endorphin, γ-endorphin, sigma-endorphin, [Leu⁵]enkephalin, [Met⁵]enkephalin, substance P), kinins (e.g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as α₁-antitrypsin, α₂-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin and combinations thereof

### RNAi

Interfering RNA (RNAi) include, e.g., antisense RNA, a ribozyme, an RNAi and an siRNA. RNAi fragments, particularly double-stranded (ds) RNAi, can be used to inhibit gene expression. One approach well known in the art for inhibiting gene expression is short interfering RNA (siRNA) mediated gene silencing, where the level of expression product of a target gene is reduced by specific double stranded siRNA nucleotide sequences that are complementary to at least a 19-25 nucleotide long segment (e.g., a 20-21 nucleotide sequence) of the target gene transcript, including the 5′ untranslated (UT) region, the ORF, or the 3′ UT region. In some embodiments, short interfering RNAs are about 19-25 nt in length. See, e.g., PCT applications WO0/44895, WO99/32619, WO01/75164, WO01/92513, WO01/29058, WO01/89304, WO02/16620, and WO02/29858; and U.S. Patent Publication No. 20040023390 for descriptions of siRNA technology. The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

Target genes include any gene encoding a target gene product (RNA or protein) that is deleterious (e.g., pathological); a target gene product that is malfunctioning; a target gene product. Target gene products include, but are not limited to, huntingtin; hepatitis C virus; human immunodeficiency virus; amyloid precursor protein; tau; a protein that includes a polyglutamine repeat; a herpes virus (e.g., varicella zoster); any pathological virus; and the like.

siRNA is useful for treating a variety of disorders and conditions, including, but not limited to, neurodegenerative diseases, e.g., a trinucleotide-repeat disease, such as a disease associated with polyglutamine repeats, e.g., Huntington's disease, spinocerebellar ataxia, spinal and bulbar muscular atrophy (SBMA), dentatorubropallidoluysian atrophy (DRPLA), etc.; an acquired pathology (e.g., a disease or syndrome manifested by an abnormal physiological, biochemical, cellular, structural, or molecular biological state) such as a viral infection, e.g., hepatitis that occurs or may occur as a result of an HCV infection, acquired immunodeficiency syndrome, which occurs as a result of an HIV infection; and the like.

In some embodiments, an siRNA is directed against a member of a signal transduction pathway, e.g., the insulin pathway, including AKT1-3, CBL, CBLB, EIF4EBP1, FOXO1A, FOXO3A, FRAP1, GSK3A, GSK3B, IGF1, IGF1R, INPP5D, INSR, IRS1, MLLT7, PDPK1, PIK3CA, PIK3CB, PIK3R1, PIK3R2, PPP2R2B, PTEN, RPS6, RPS6KA1, RPX6KA3, SGK, TSC1, TSC2, and XPO1); an apoptotic pathway (CASP3,6,7,8,9, DSH1/2, P110, P85, PDK1/2, CATENIN, HSP90, CDC37, P23, BAD, BCLXL, BCL2, SMAC, and others); and pathways involved in DNA damage, cell cycle, and the like (p53, MDM2, CHK1/2, BRCA1/2, ATM, ATR, P151NK4, P27, P21, SKP2, CDC25C/A, 14-3-3, PLK, RB, CDK4, GLUT4, Inos, Mtor, FKBP, PPAR, RXR, ER). Similarly, genes involved in immune system function including TNFR1, IL-IR, IRAK1/2, TRAF2, TRAF6, TRADD, FADD, IKKε, IKKγ, IKKβ, IKKα, IkBα, IkKβ, p50, p65, Rao, RhoA, Cdc42, ROCK, Pak1/2/3/4/5/6, cIAP, HDAC1/2, CBP, β-TrCP, R1/4, and others are also important targets for siRNAs, where such siRNAs can be useful in treating immune system disorders. siRNAs specific for gene products involved in apoptosis, such as Dsh1/2, PTEN, P110 (pan), P85, PDK1/2, Akt1, Akt2, Akt (pan), p70^{S6K}, GSK3β, PP2A (cat), β-catenin, HSP90, Cdc37/p50, P23, Bad, Bc1xL, Bc12, Smac/Diablo, and Ask1 are useful in the treatment of diseases that involve defects in programmed cell death (e.g. in the treatment of cancer). siRNA agents directed against p53, MDM2, Chk1/2, BRCA1/2, ATM, ATR, p15^{INK4}, P27, P21, Skp2, Cdc25C/A, 14-3-3sigma/ε, PLK, Rb, Cdk4, Glut4, iNOS, mTOR, FKBP, PPARγ, RXRα, ERα, and related genes can be used to treat diseases associated with disruptions in DNA repair, and cell cycle abnormalities, where such diseases include cancer. Examples of such siRNAs and targets are known in the art; see, e.g., US Patent Publication No. 2005/0246794.

As such a subject recombinant retroviral vector that includes a heterologous nucleic acid encoding an siRNA is useful for treating disorders resulting from or associated with dysregulated cell cycle, e.g., cancer.

### Methods of Making a Microsphere

The Example, below, provides a non-limiting example of how to make a subject microsphere.

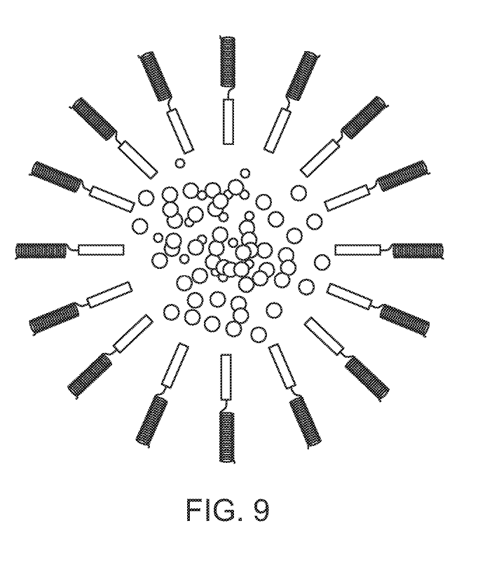

A schematic depiction of a nanoparticle (or microparticle) within a subject microsphere is provided in FIG. 9. In FIG. 9, the innermost dots represent the drug molecules contained within the micelle. The polymer-drug micelle is formed by the hydrophobic forces driving the polylactide chains (inner rods) and the drug molecules to the hydrophobic core while leaving the mpEG chains (outer rods) exposed to the aqueous hydrophilic environment. Both the mpEG and pLL chain length may be varied by changing reaction conditions. The fabrication of the pLL-mpEG copolymer have been previously described (Park and Healy, 2003 supra). Briefly, ring opening polymerization of cyclic L-lactide (1,4-dioxane-2,5-dione) is performed on methoxy-poly(ethylene glycol)-hydroxyl (mpEG, MW=3000) for 6 h at 95° C., using tin (II) 2-ethylhexanoate (Sn(Oct)₂) as catalyst. The pLL chain length is controlled by changing lactide monomer:hydroxyl ratios (M/OH). Further variations include grafting of polylysine to the inner polylactide chain by first succinylating the hydroxyl end group of polylactide to be amine reactive.

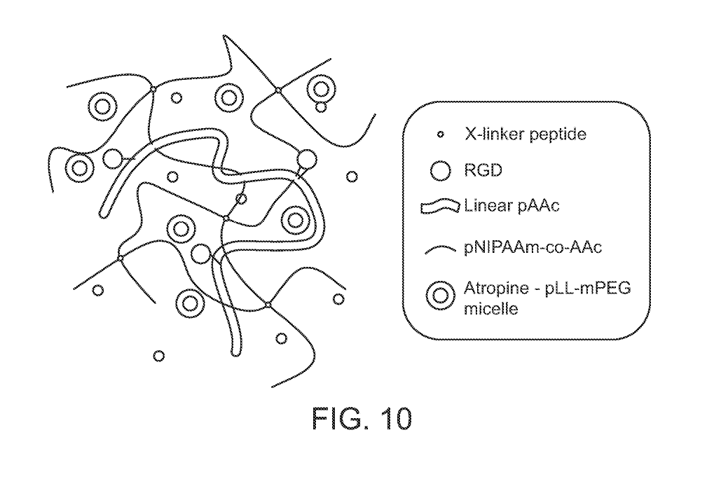

FIG. 10 presents a schematic depiction of various embodiments of a subject microsphere. For example, the x-linker peptide could be any metalloproteinase degradable crosslinker previously described (Kim and Healy, 2003, supra), or could simply be N,N′-methylenebis(acrylamide) crosslinker The cell peptide RGD could also be switched to other types according to the local cellular environment.

One method of incorporating the micelles (e.g., nanoparticles; microparticles) into the hydrogel is by simple absorption (passive diffusion) into a rehydrating hydrogel. First, the hydrogel is lyophilized, then immersed in media that contains the formed micelles. For commercial applications, the micelles can be packaged as lyophilized powder, while the hydrogel is packaged in hydrated form. Upon use, the micelles are exposed to hydrogel in an aqueous media, and sufficient time is allowed for homogeneous absorption into the hydrogel prior to injection. An alternative method is by physically mixing into the gel solution as the hydrogel is formed, thus homogeneously embedding the micelles into the hydrogel matrix. Using this method, the hydrogel and micelle system would be packaged together in hydrated form.

### Utility

A subject microsphere is useful in a variety of diagnostic and therapeutic applications, which are also provided.

A subject microsphere can be formulated with one or more pharmaceutically acceptable excipients. A wide variety of pharmaceutically acceptable excipients are known in the art and need not be discussed in detail herein Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Unit dosage forms of a subject microsphere for injection or intravenous administration may comprise a subject microsphere in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of a subject microsphere comprising an active agent calculated in an amount sufficient to produce the desired effect in association with a pharmaceutically acceptable diluent, carrier or vehicle. The specifications for a subject microsphere depend on the particular active agent contained within the microsphere and the effect to be achieved, and the pharmacodynamics associated with each compound in the host.

A unit dosage form of a subject microsphere can include from about 10⁵ to about 10⁹ microspheres, where a unit dosage form of a subject microsphere comprises from about 1 ng to about 10 mg of an active agent.

In some embodiments, multiple doses of a subject microsphere composition are administered. The frequency of administration of a subject microsphere composition can vary depending on any of a variety of factors, e.g., severity of the symptoms, etc. For example, in some embodiments, a subject compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

A subject microsphere composition is administered to an individual using any available method and route suitable for drug delivery, including in vivo and ex vivo methods, as well as systemic and localized routes of administration. Administration can be acute (e.g., of short duration, e.g., a single administration, administration for one day to one week), or chronic (e.g., of long duration, e.g., administration for longer than one week, e.g., administration over a period of time of from about 2 weeks to about one month, from about one month to about 3 months, from about 3 months to about 6 months, from about 6 months to about 1 year, or longer than one year).

Conventional and pharmaceutically acceptable routes of administration include intranasal, intramuscular, intratracheal, subcutaneous, intradermal, transdermal, sublingual, ocular, intraorbital, topical application, intravenous, rectal, nasal, oral, and other enteral and parenteral routes of administration. Routes of administration may be combined, if desired, or adjusted depending upon the agent and/or the desired effect.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Delivery of siRNA

This report describes the synthesis and characterization of poly(lysine-g-(lactide-b-ethylene glycol)) terpolymers for subsequent nanoparticulate packaging of siRNA. The positively charged polylysine (pK) core in the polymer comb serves to promote siRNA binding and condensation. In the grafted block copolymer, poly(ethylene glycol) (pEG) provides an uncharged hydrophilic shell that improves particle colloidal stability and prevents undesired protein adsorption. The intermediate hydrophobic poly-L-lactide (pLL) enhances complex stability in aqueous environment, facilitates premature siRNA condensation through hydrophobic interactions, and protects siRNA from intracellular degradation. Through the hydrolytic degradation of ester backbone, pLL also provides a mechanism of controlled siRNA release profile. Park, S. Healy, K. E. Nanoparticulate DNA packaging using terpolymers of poly(lysine-g-(lactide-b-ethylene glycol)). Bioconjugate Chem (2003) 14: 31119. The aim of this project is to modulate the molecular weight of the pLL and/or pK segments to achieve zero-order kinetics of siRNA delivery.

### Materials and Methods

### Diblock Copolymer Synthesis

The copolymer is synthesized by ring opening polymerization of L-lactide (1,4-dioxane-2,5-dione). L-lactide was purified by recrystallization from toluene and vacuum dried for at least 1 hour immediately before use. Methoxy-poly(ethylene glycol)-hydroxyl (mpEG, MW=3000) was dried under nitrogen overnight. The reaction was prepped in a sealed dry nitrogen atmosphere. The L-lactide and mpEG were reacted in anhydrous toluene at monomer:hydroxyl ratios (M/OH) of 40 and 60 to alter pLL molecular weights. A solution of 0.5M stannous octoate in anhydrous toluene was prepared at a lactide monomer:initiator ratio (M/I) of 300, and introduced to the L-lactide-mpEG mixture to initiate polymerization.

Polymerization proceeded in refluxing toluene at 95° C. under a gentle flow of dry nitrogen. Toluene reflux was maintained with a chiller pumping an ethylene glycol-water mixture at 4° C. After 6 hours of reaction, the system was cooled to room temperature. The polymerized slurry was purified by repeated dissolution in dioxane and reprecipitation into excess ice-cold diethyl ether. The pLL-mpEG copolymer product was dried overnight under dry nitrogen and stored at −20° C.

### Succinylation of Lactide Hydroxyl Group

The lactide hydroxyl end group of the pLL-mpEG copolymer was functionalized by succinylation to be amine reactive. 0.6M of the copolymer in anhydrous dioxane was added to a 6-fold molar excess each of 0.35M disuccinimidyl carbonate (DSC) and 0.35M base catalyst 4-dimethylamino pyridine (DMAP) in anhydrous dimethylformamide (DMF). The reaction was prepped in sealed dry nitrogen atmosphere. The mixture was reacted at room temperature under stirring in dry nitrogen for 6 hours, after which the product was purified by repeated dissolution in DMF and reprecipitation into diethyl ether. The final product (su-pLL-mpEG) was dried overnight under dry nitrogen and stored at −20° C.

### Grafting of Polylysine

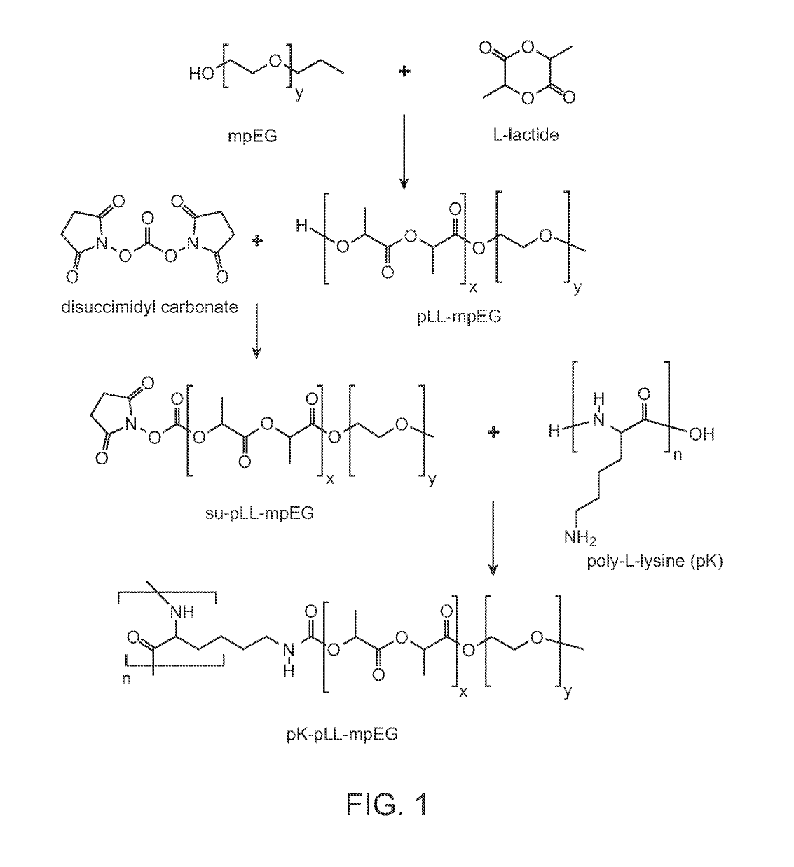

Grafting onto polylysine was achieved by reacting su-pLL-mpEG with the E-amines on poly-L-lysine (pK) (Mw=8800, DP=42). A ˜25 mg/ml solution of su-pLL-mpEG in 1:1 dimethyl sulfoxide (DMSO) and DMF was added to a ˜15 mg/ml solution of pK in DMSO. The reaction was prepped in sealed dry nitrogen atmosphere. The mixture was reacted at 1:1 molar ratio at room temperature under stirring in dry nitrogen for 6 hours, after which solvent was removed by freeze drying overnight. Purification was done by sequential washing in tetrahydrofuran (THF) and methanol and reprecipitation into diethyl ether. The final product was dried using a rotator evaporator and stored at minus 20° C. The overall terpolymer synthesis scheme is represented in FIG. 1.

### Characterization

Size Exclusion Chromatography with Multiangle Laser Light Scattering (SECMALLS)

Molecular weights and polydispersity index of the pLL-mpEG copolymer were determined by SEC-MALLS, using acetonitrile as the mobile phase. The system consists of an Agilent 1100 HPLC, connected in series with a Wyatt Optilab refractive interferometer and DAWN E MALLS detector. Using the refractive interferometer, the dn/dc values of each copolymer in acetonitrile were experimentally measured from serial dilutions of known concentrations. Data were analyzed with the Wyatt DNDC and ASTRA software. Molecular weights were determined from linear Debye plots.

### Critical Micelle Concentration (CMC)

The CMC of the ampiphilic pLL-mpEG diblock copolymer was measured by dye micellization method using Eosin Y. The absorbance peak of Eosin Y shifts from 518 nm in water to 542 nm in high surfactant environment [9]. Varying concentrations of pLL-mpEG emulsion was obtained by serially diluting the copolymer in DMSO and mixing into ultrapure water in 1:9 v/v ratio. The final concentration of Eosin Y in all mixtures was kept constant at 0.019 mM. CMC was determined by tracking changes in the absorbance of the micellized Eosin Y dye at a constant wavelength of 542 nm in varying copolymer concentrations. All measurements were taken in triplicates (n=3). The absorbance versus concentration (c) plot was fitted by least square analysis in Matlab into a logistic function below:

$$Absorbance ( c ) = \frac{K}{1 + exp [ - r ( c - {c}_{o} ) ]} + background \qquad ( 1 )$$

K, r and co are constants. The concentration at the inflection point of the fitted logistic curve was taken to be the CMC.

### Proton Nuclear Magnetic Resonance Spectroscopy (¹H NMR)

The products of the three synthesis steps were characterized using ¹H NMR on a Bruker AVQ-400, using deuterated dimethyl sulfoxide (DMSO-d6) as a solvent. Based on pLL:pEG and pLL:pK peak ratios, ¹H NMR was also used to calculate the number-averaged molecular weight of pLL:mpEG copolymer and percent substitution (grafting) of pK in the terpolymer.

### Results

Size Exclusion Chromatography with Multiangle Laser Light Scattering (SECMALLS)

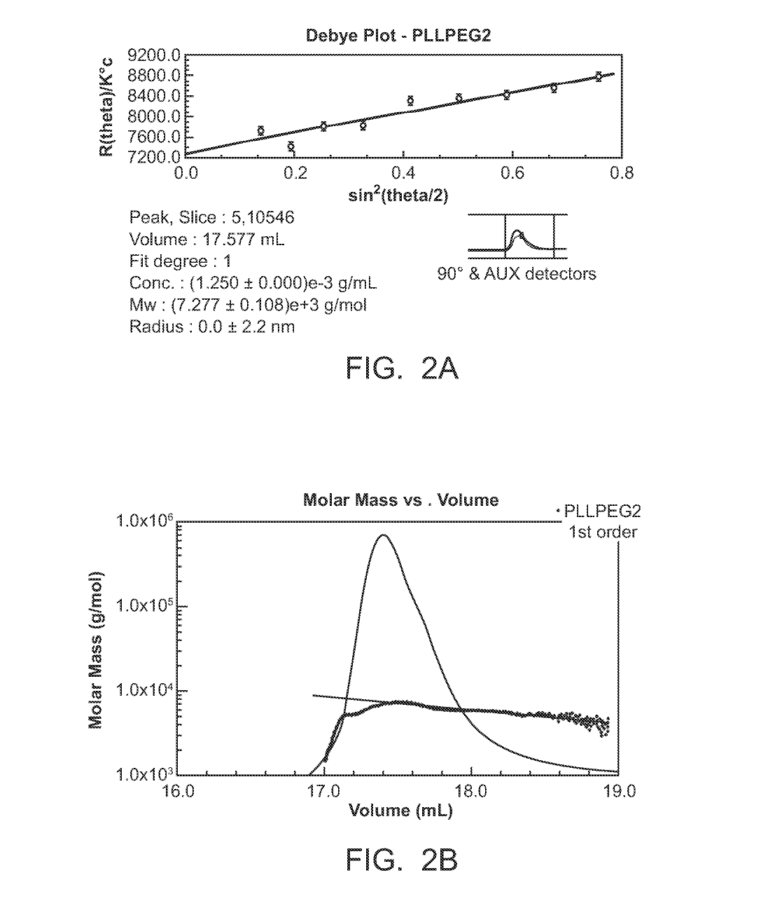

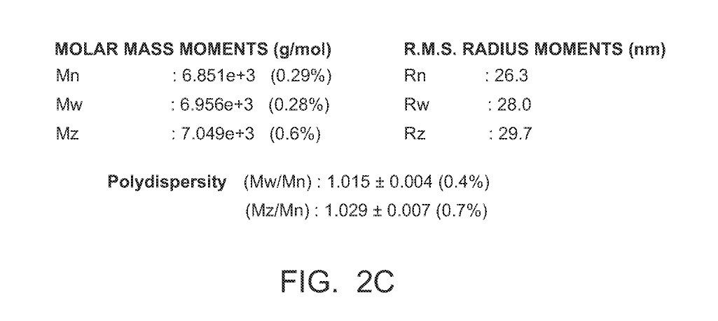

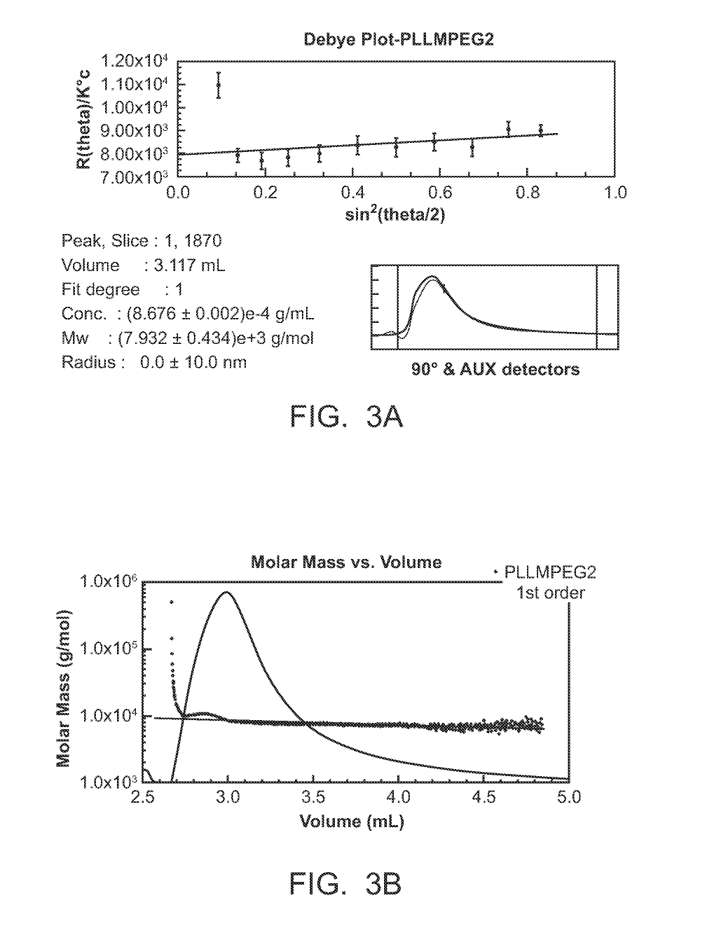

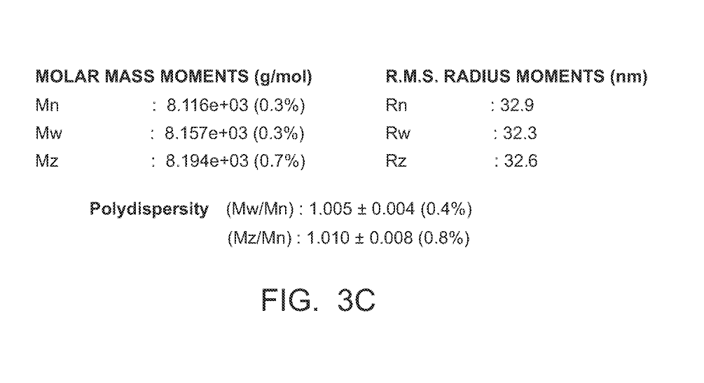

In the ring opening polymerization reaction, increasing lactide monomer:hydroxyl (M/OH) ratio from 40 to 60 caused the dn/dc value of pLL-mpEG in acetonitrile to decrease from 0.095 mL/g to 0.077 mL/g, the number-averaged molecular weight to increase from 6850 g/mol to 8120 g/mol, and the number-averaged radius of gyration to increase from 26.3 nm to 31.9 nm. The polymerization reactions produced narrow molecular weight distributions, with polydispersity indices below 1.1. The SEC-MALLS traces of the pLL-mpEG copolymers are shown in FIGS. 2A-C and FIGS. 3A-C.

FIGS. 2A-C. SEC-MALLS data of pLL-mpEG reacted from lactide monomer:hydroxyl (M/OH) ratio of 40, showing a) a representative linear Debye plot; b) a molar mass distribution plot; and c) numerical molecular weight and radius gyration data. FIGS. 3A-C. SEC-MALLS data of pLL-mpEG reacted from lactide monomer:hydroxyl (M/OH) ratio of 60, showing a) a representative linear Debye plot; b) a molar mass distribution plot; and c) numerical molecular weight and radius gyration data.

### Critical Micelle Concentration (CMC)

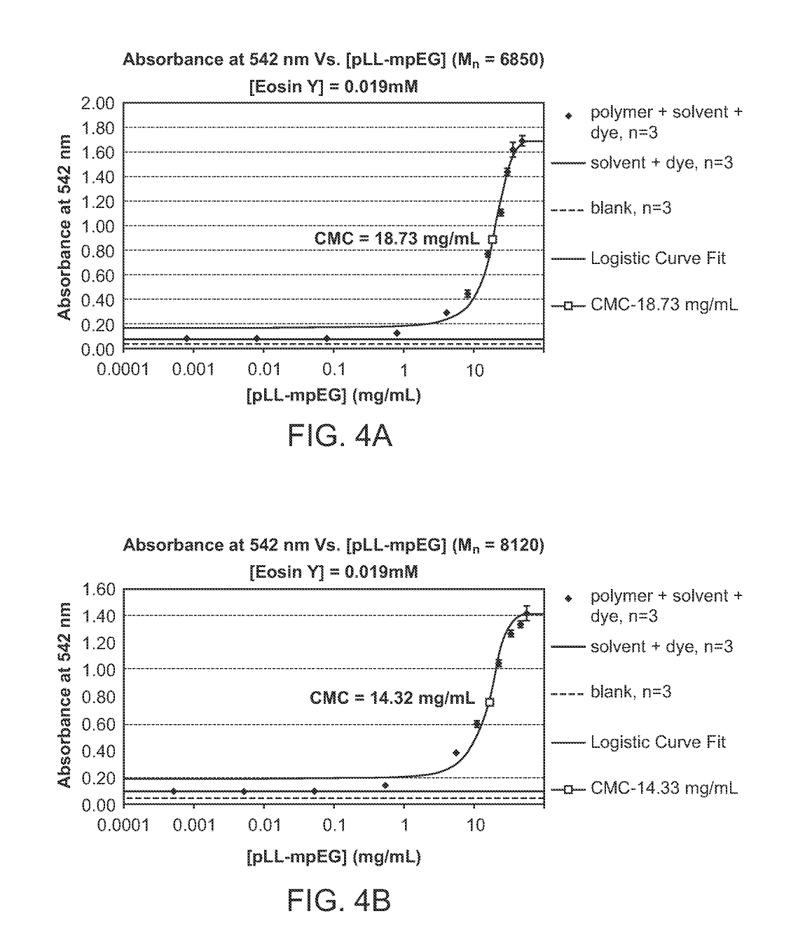

CMC values of pLL-mpEG of Mn 6850 g/mol and 8120 g/mol (reacted from M/OH ratios of 40 and 60) are 18.73 mg/mL and 14.32 mg/mL respectively. Keeping the pEG segment constant, increasing the length of the pLL segment causes the hydrophobic nature of the ampiphilic copolymer to increase. Being less stable in aqueous solution, the more hydrophobic copolymer tends to micellize at lower concentrations. Consequently, CMC decreases with increasing pLL chain length. The absorbance data, as well as their fit into the logistic curves are shown in FIGS. 4A and B.

FIGS. 4A and B. Eosin Y absorbance data for the CMC determination of pLL-mpEG with molecular weights: a) Mw=6850 g/mol and b) Mw=8120 g/mol.

### Proton Nuclear Magnetic Resonance Spectroscopy (¹H NMR)

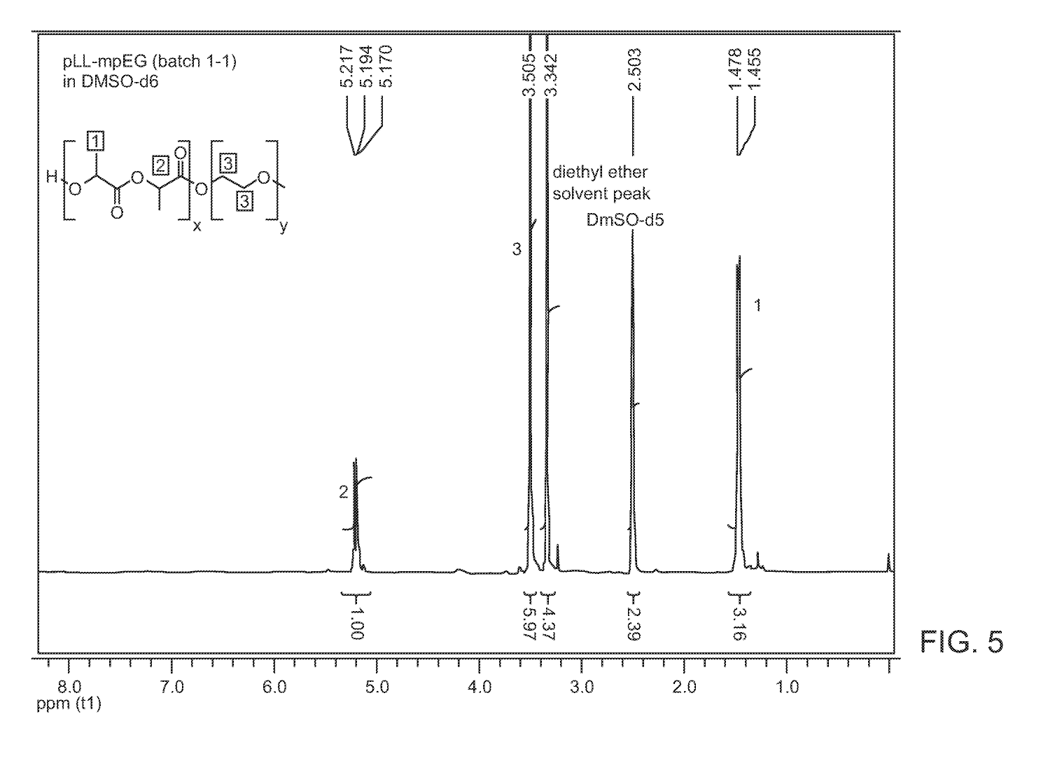

The ¹H NMR traces from the first terpolymer synthesis (shown in FIGS. 5-7) display relevant peaks after each synthesis step, indicating successful reaction and purification procedures although the presence of a peak at 3.34 ppm for both pLL-mpEG and su-pLL-mpEG reveals residual diethyl ether solvent from the final reprecipitation step. However, since diethyl ether is unreactive, this residual solvent is not expected to affect the stability or the reactivity of the copolymers. The number average molecular weight calculated using the peak ratio of pLL (peak 2) to pEG (peak 3) was determined to be 6330 g/mol, which is in close agreement with that obtained from SEC-MALLS (Mn=6850 g/mol). Based on pLL (peak 2):pK (peak 6) ratio, 16.4% of the E-amines in the lysine residues was substituted with pLL-mpEG.

The ¹H NMR spectra are also consistent with those obtained from the same terpolymer system reported by S. Park [8].

FIG. 5: ¹H NMR spectrum of pLL-mpEG (Mn=6850 g/mol).

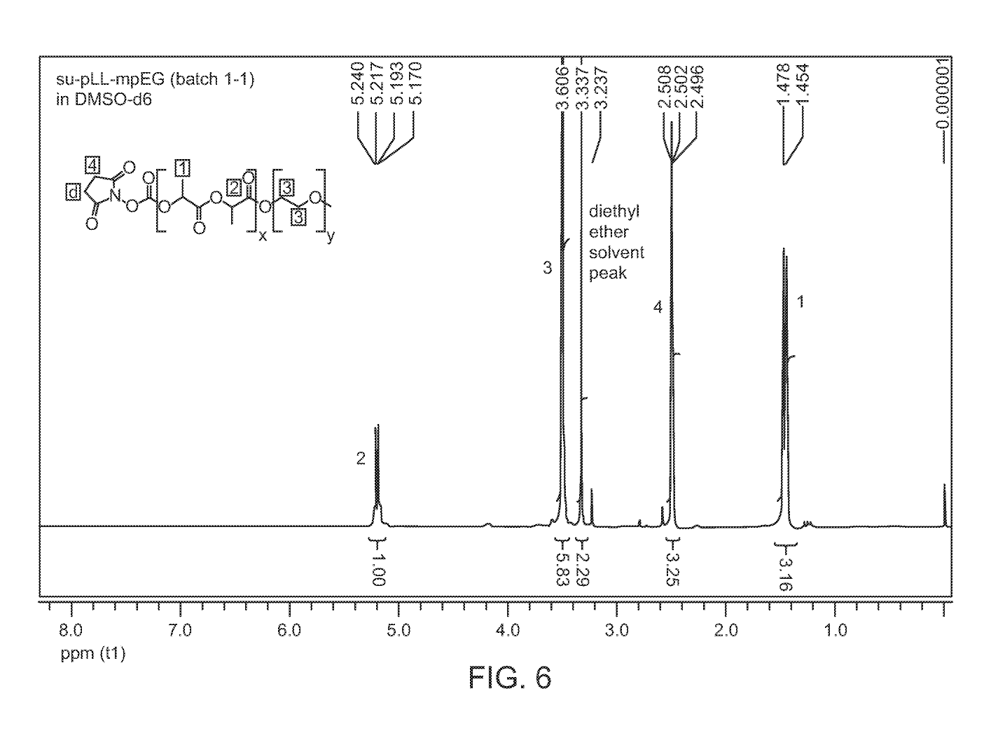

FIG. 6: ¹H NMR spectrum of su-pLL-mpEG.

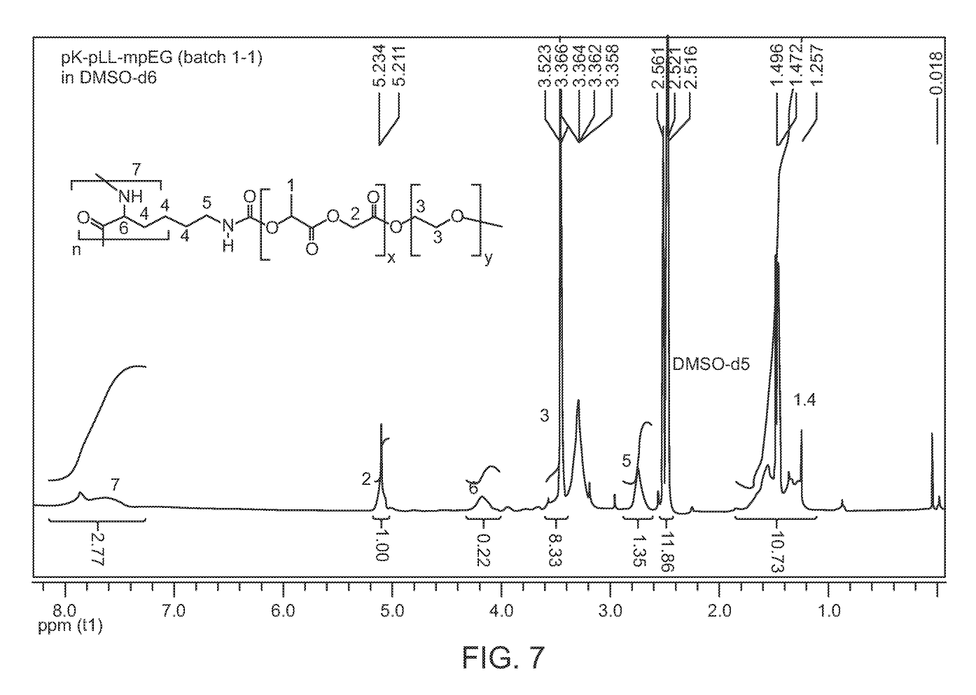

FIG. 7: ¹H NMR spectrum of pK-pLL-mpEG terpolymer.

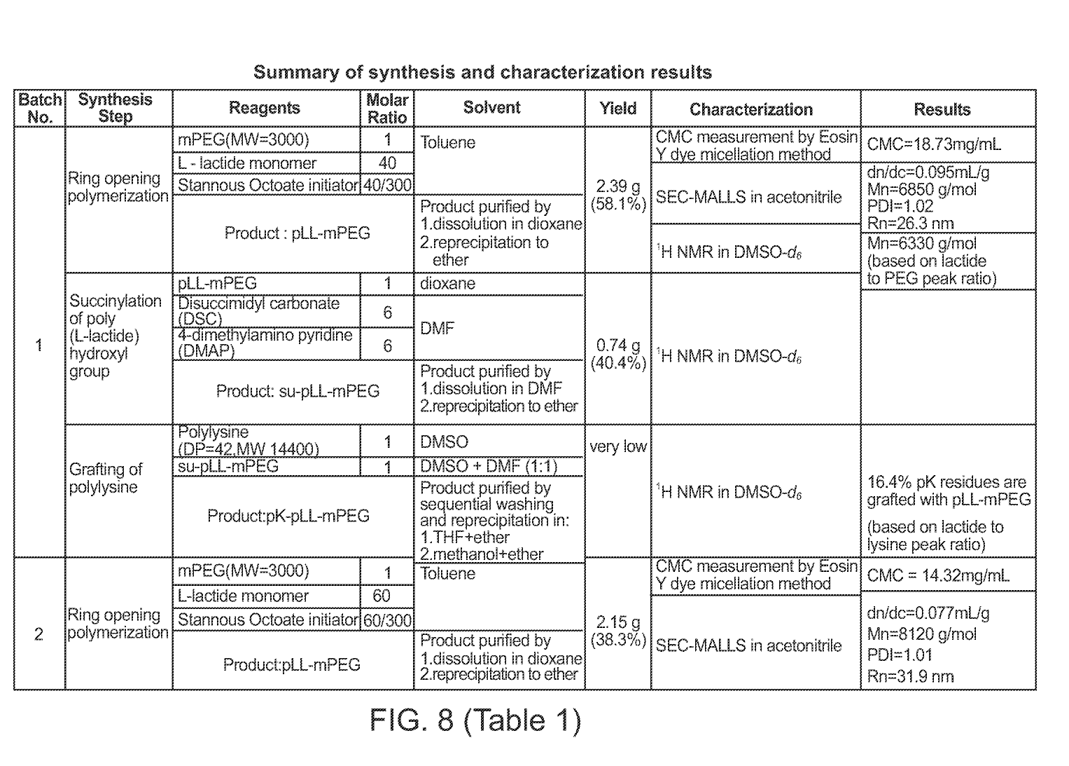

The summary of experimental data is provided in FIG. 8 (Table 1).

### Example 2

### Ocular Delivery of an Active Agent

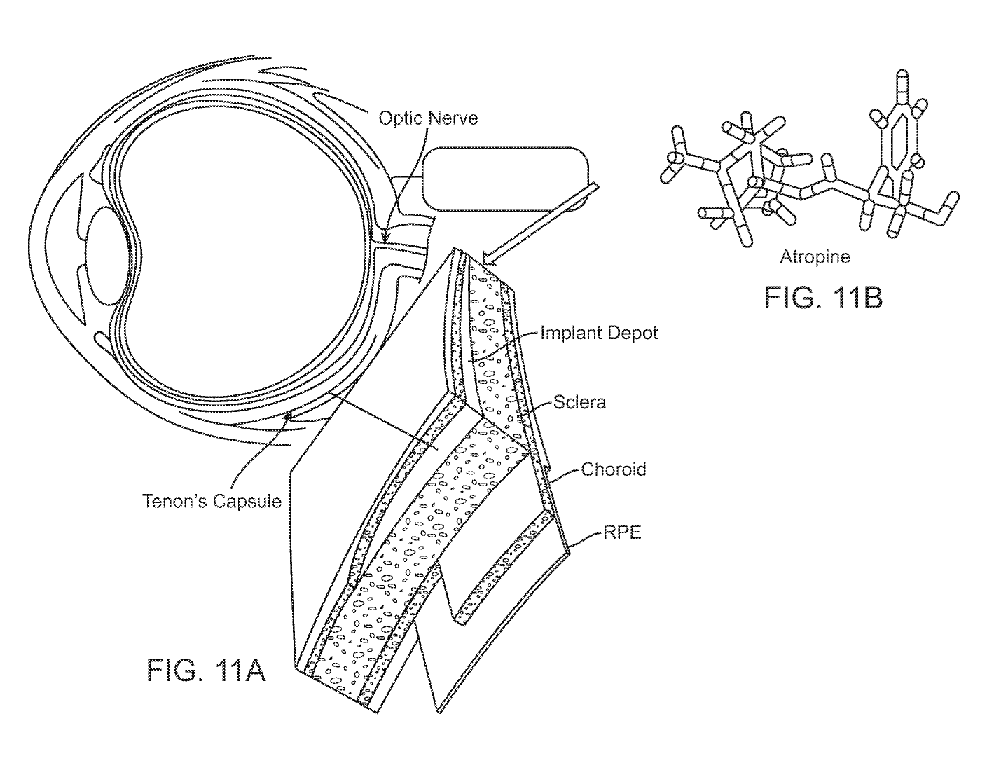

General features of various aspects of the invention, as well as examples relating to ocular delivery, are presented in FIGS. 11-19.

FIG. 11A depicts ocular drug delivery of atropine. As an example, a subject drug delivery system is injected as a sub-Tenon's implant at the posterior pole of the eye. Transscleral drug delivery can be achieved by this method. FIG. 11B depicts the structure of atropine.

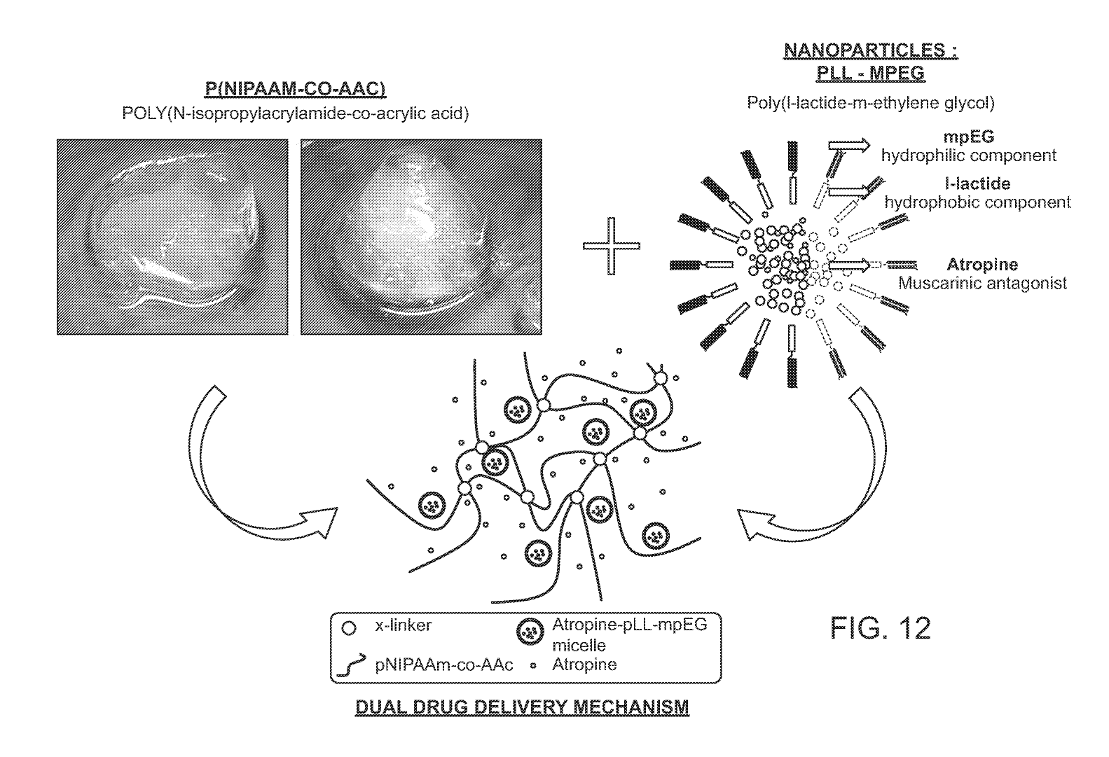

FIG. 12 depicts poly(N-isopropylacrylamide-co-acrylic acid) (pNIPAAM-Co-AAC) hydrogel and poly(L-lactide)-methoxy-poly(ethylene glycol)) (pLL-MPEG) nanoparticles. The thermal properties of pNIPAAM-Co-AAC hydrogel are depicted in the left and right panels. The left panel shows that at room temperature, the hydrogel is a transparent viscous gel; the right panel shows that above 37° C., the hydrogel becomes opaque and becomes stiffer. Combining the pNIPAAM-Co-AAC hydrogel and pLL-MPEG nanoparticles (in which atropine is encapsulated) provides for a dual-release drug delivery system.

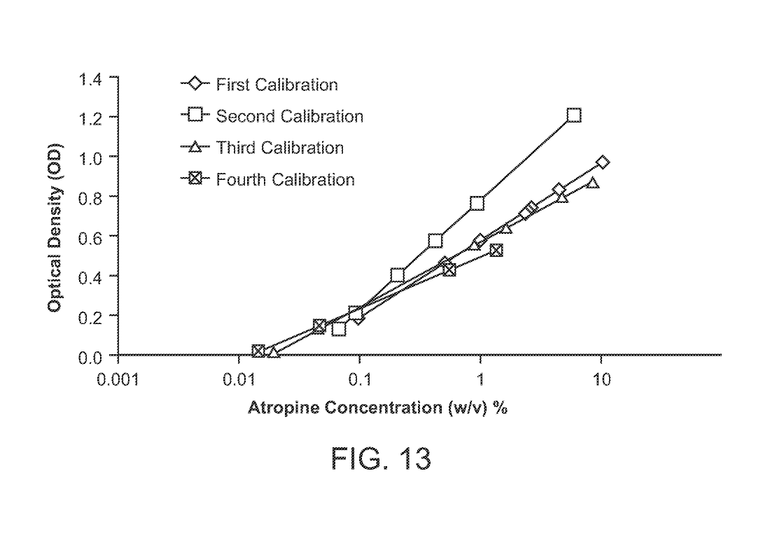

FIG. 13 depicts optical density vs. atropine concentration for atropine solutions.

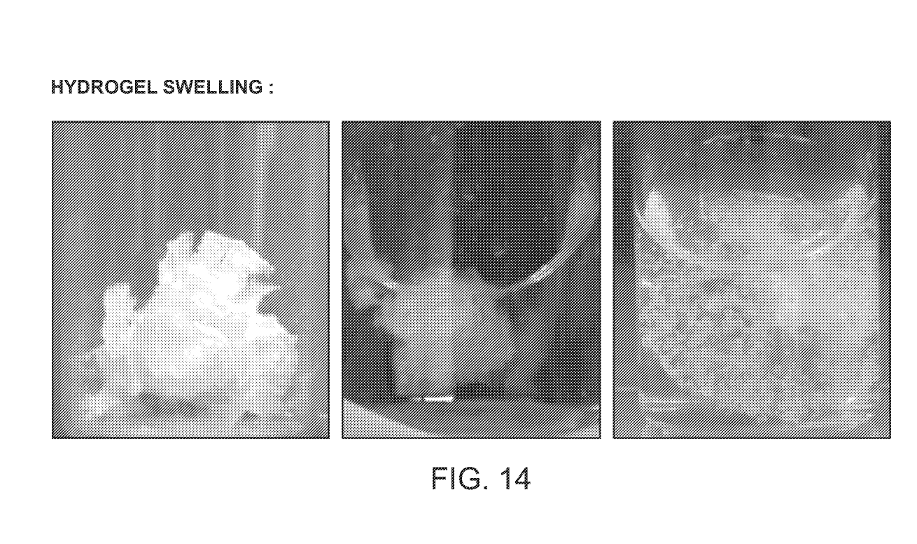

FIGS. 14A-C depict the swelling capacity of hydrogel. FIG. 14A depicts freeze-dried hydrogel; FIG. 14B depicts the hydrogel in 1% atropine solution at time zero (1=0); and FIG. 14C depicts fully swelled hydrogel at t=96 hours.

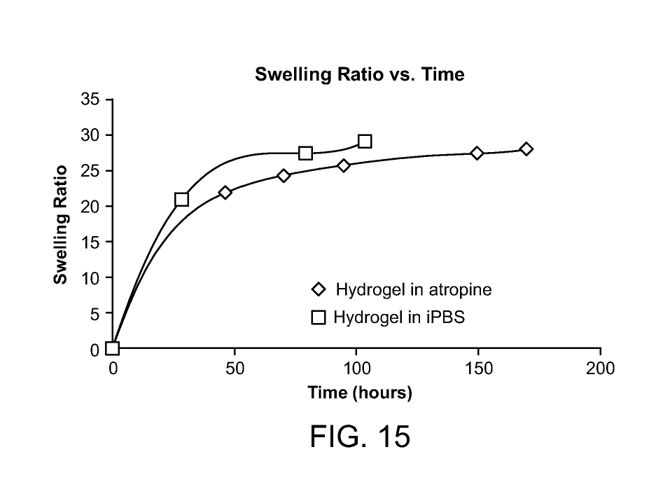

FIG. 15 depicts swelling variation with time and media. The swelling ratio was found to depend on the ratio of the components, the extent of dehydration, and the medium in which the hydrogel was placed. The hydrogel swelled more in isotonic phosphate buffered saline (iPBS), compared to the atropine solution.

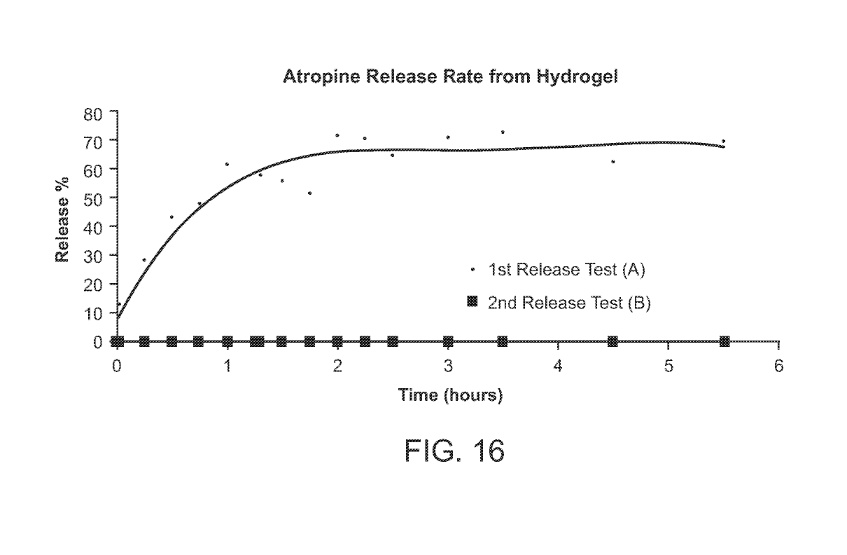

FIG. 16 depicts release rate of atropine from hydrogel. The first preliminary testing (A) of atropine released from hydrogel showed that the hydrogel absorbed all of the drug solution in which the hydrogel was soaked, and that the hydrogel release the atropine solution when place in medium at 37° C. and static conditions. Experimental numbers illustrate a 68% release, most of which occurred during the first two hours of study, indicated that there was a burst effect. Hydrogel for the second release test (B) was soaked in excess drug solution to reach maximum swelling. Almost no drug retention was observed; therefore, the release is approximately zero.

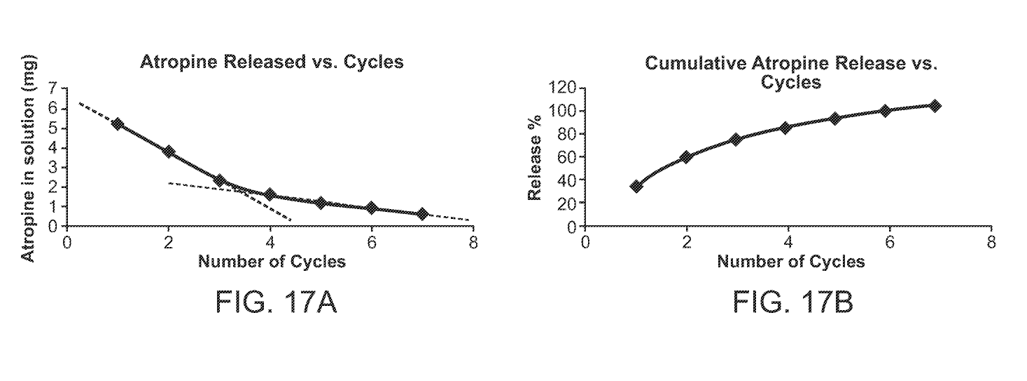

FIGS. 17A and 17B depict atropine release. FIG. 17A: The analysis of atropine content in filtrate per cycle showed two different release behaviors. The first sharp decrease in atropine release would be related to the excess atropine in solution. The slow release characteristic of the second slope would be the drug release associated with nanoparticle degradation. FIG. 17B: Cumulative release percentage per cycle shows that all the atropine was release by the seventh cycle.

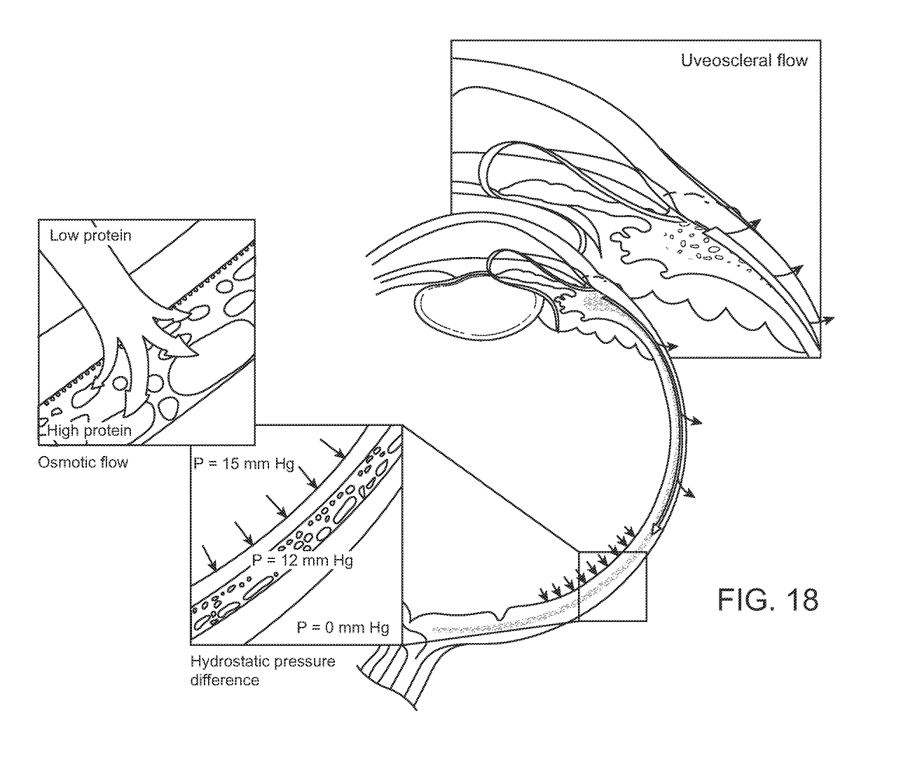

FIG. 18 schematically depicts transscleral drug delivery. A transscleral drug delivery system requires the drug to permeate through multiple ocular tissues and fluid pressure gradients in order to reach the neuro-retina.

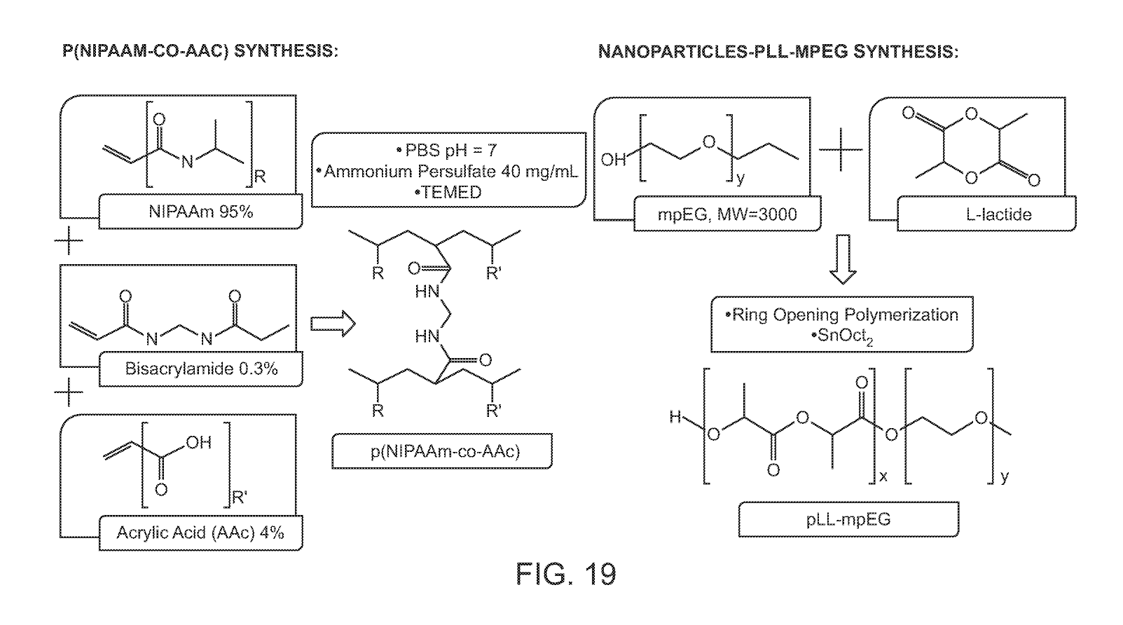

FIG. 19 depicts poly(N-isopropylacrylamide-co-acrylic acid) hydrogel and poly(L-lactide-m-ethylene glycol) synthesis. Poly(NIPAAM-Co-AAC) synthesis is depicted, in which N-isopropylacylamide (NIPAAM) (95%), bisacrylamide (0.3%), and acrylic acid (45) are reacted in a solution of phosphate buffered saline (pH=7), ammonium persulfate (40 mg/ml) and N,N,N,N-Tetramethylethylenediamine (TEMED) to yield p(NIPAAM-Co-AAC). Nanoparticle synthesis (pLL-mPEG synthesis) is depicted. Methoxy-poly(ethylene glycol)-hydroxyl (mpEG, MW=3000) and L-lactide are reacted using Sn(II)₂-ethylhexanoate (Sn(Oct)₂) to initiate polymerization.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A biodegradable microsphere comprising:
a) a nanoparticle comprising an active agent; and
b) a hydrogel surrounding the nanoparticle.

2. The microsphere of claim 1, wherein the nanoparticle comprises a hydrophobic polymer and a hydrophilic polymer, wherein the hydrophobic polymer forms a hydrophobic core.

3. The microsphere of claim 2, wherein the hydrophilic polymer is a poly(ethylene glycol) polymer.

4. The microsphere of claim 2, wherein the hydrophobic polymer is poly(L-lactide).

5. The microsphere of claim 2, wherein the active agent is not linked to a nanoparticle polymer.

6. The microsphere of claim 2, wherein active agent is linked to the hydrophobic polymer or the hydrophilic polymer.

7. The microsphere of claim 1, wherein the active agent is hydrophobic.

8. The microsphere of claim 1, wherein the hydrogel comprises poly(N-isopropylacrylamide-co-acrylic acid).

9. The microsphere of claim 8, wherein the active agent in the nanoparticle is a first active agent, and wherein the hydrogel comprises a second active agent that is different from the first active agent.

10. The microsphere of claim 9, wherein the second active agent is a hydrophilic agent.

11. The microsphere of claim 8, wherein the first active agent is released at a first rate and over a first time period, and the second active agent is released at a second rate and over a second time period.

12. The microsphere of claim 1, wherein the hydrogel comprises a crosslinking peptide comprising a proteolytic cleavage site.

13. The microsphere of claim 1, wherein the hydrogel comprises a cell surface receptor binding moiety.

14. The microsphere of claim 1, wherein the active agent is a small molecule drug, a polypeptide, or a nucleic acid.

15. The microsphere of claim 14, wherein the nucleic acid is an interfering RNA.

16. A composition comprising:
a) a microsphere of claim 1; and
b) a buffer.

17. A pharmaceutical composition comprising:
a) a microsphere of claim 1; and
b) a pharmaceutically acceptable excipient.

18. A method of delivering an active agent to an individual in need thereof, the method comprising administering the pharmaceutical composition of claim 17 to the individual.

19. A method of treating a disorder in an individual in need thereof, the method comprising administering an effective amount of the pharmaceutical composition of claim 17 to the individual.


SEQUENCE LISTING (43 sequences)

SEQ ID NO: 1 - protein, 15 aa, Artificial Sequence
  Synthetic Peptide
CGGNGEPRGDTYRAY

SEQ ID NO: 2 - protein, 7 aa, Artificial Sequence
  Synthetic Peptide
FHRRIKA

SEQ ID NO: 3 - protein, 15 aa, Artificial Sequence
  Synthetic Peptide
CGGNGEPRGDTYRAY

SEQ ID NO: 4 - protein, 10 aa, Artificial Sequence
  Synthetic Peptide
CGGFHRRIKA

SEQ ID NO: 5 - protein, 12 aa, Artificial Sequence
  Synthetic Peptide
CEPRGDTYRAYG

SEQ ID NO: 6 - protein, 12 aa, Artificial Sequence
  Synthetic Peptide
CGGGEAPRGDVY

SEQ ID NO: 7 - protein, 10 aa, Artificial Sequence
  Synthetic Peptide
CCGPRGDVYG

SEQ ID NO: 8 - protein, 18 aa, Artificial Sequence
  Synthetic Peptide
CGGVSWFSRHRYSPFAVS

SEQ ID NO: 9 - protein, 15 aa, Artificial Sequence
  Synthetic Peptide
CGGNRWHSIYITRFG

SEQ ID NO: 10 - protein, 15 aa, Artificial Sequence
  Synthetic Peptide
CGGTWYKIAFQRNRK

SEQ ID NO: 11 - protein, 15 aa, Artificial Sequence
  Synthetic Peptide
CGGRKRLQVQLSIRT

SEQ ID NO: 12 - protein, 15 aa, Artificial Sequence
  Synthetic Peptide
CGGKAFDITYVRLKF

SEQ ID NO: 13 - protein, 10 aa, Artificial Sequence
  Synthetic Peptide
CTRKKHDNAQ

SEQ ID NO: 14 - protein, 15 aa, Artificial Sequence
  Synthetic Peptide
VSWFSRHRYSPFAVS

SEQ ID NO: 15 - protein, 10 aa, Artificial Sequence
  Synthetic Peptide
RNIAEIIKDI

SEQ ID NO: 16 - protein, 12 aa, Artificial Sequence
  Synthetic Peptide
TAGSCLRKFSTM

SEQ ID NO: 17 - protein, 10 aa, Artificial Sequence
  Synthetic Peptide
TTSWSQCSKS

SEQ ID NO: 18 - protein, 13 aa, Artificial Sequence
  Synthetic Peptide
RYVVLPRPVCFEK

SEQ ID NO: 19 - protein, 5 aa, Artificial Sequence
  Synthetic Peptide
EVLLI

SEQ ID NO: 20 - protein, 6 aa, Artificial Sequence
  Synthetic Peptide
PXGMTS

SEQ ID NO: 21 - protein, 5 aa, Artificial Sequence
  Synthetic Peptide
PXGMT

SEQ ID NO: 22 - protein, 10 aa, Artificial Sequence
  Synthetic Peptide
CGLVPAGSGP

SEQ ID NO: 23 - protein, 12 aa, Artificial Sequence
  Synthetic Peptide
SLLKSRMVPNFN

SEQ ID NO: 24 - protein, 12 aa, Artificial Sequence
  Synthetic Peptide
SLLIARRMPNFN

SEQ ID NO: 25 - protein, 12 aa, Artificial Sequence
  Synthetic Peptide
SKLVQASASGVN

SEQ ID NO: 26 - protein, 12 aa, Artificial Sequence
  Synthetic Peptide
SSYLKASDAPDN

SEQ ID NO: 27 - protein, 12 aa, Artificial Sequence
  Synthetic Peptide
RPKPQQFFGLMN

SEQ ID NO: 28 - protein, 12 aa, Artificial Sequence
  Synthetic Peptide
SLRPLALWRSFN

SEQ ID NO: 29 - protein, 12 aa, Artificial Sequence
  Synthetic Peptide
SPQGIAGQRNFN

SEQ ID NO: 30 - protein, 14 aa, Artificial Sequence
  Synthetic Peptide
DVDERDVRGFASFL

SEQ ID NO: 31 - protein, 12 aa, Artificial Sequence
  Synthetic Peptide
SLPLGLWAPNFN

SEQ ID NO: 32 - protein, 12 aa, Artificial Sequence
  Synthetic Peptide
SLLIFRSWANFN

SEQ ID NO: 33 - protein, 12 aa, Artificial Sequence
  Synthetic Peptide
SGVVIATVIVIT

SEQ ID NO: 34 - protein, 12 aa, Artificial Sequence
  Synthetic Peptide
KKSPGRVVGGSV

SEQ ID NO: 35 - protein, 12 aa, Artificial Sequence
  Synthetic Peptide
PQGLLGAPGILG

SEQ ID NO: 36 - protein, 31 aa, Artificial Sequence
  Synthetic Peptide
HGPEGLRVGFYESDVMGRGHARLVHVEEPHT

SEQ ID NO: 37 - protein, 12 aa, Artificial Sequence
  Synthetic Peptide
GPQGLAGQRGIV

SEQ ID NO: 38 - protein, 12 aa, Artificial Sequence
  Synthetic Peptide
GGSGQRGRKALE

SEQ ID NO: 39 - protein, 12 aa, Artificial Sequence
  Synthetic Peptide
SLSALLSSDIFN

SEQ ID NO: 40 - protein, 12 aa, Artificial Sequence
  Synthetic Peptide
SLPRFKIIGGFN

SEQ ID NO: 41 - protein, 12 aa, Artificial Sequence
  Synthetic Peptide
SLLGIAVPGNFN

SEQ ID NO: 42 - protein, 12 aa, Artificial Sequence
  Synthetic Peptide
FFKNIVTPRTPP

SEQ ID NO: 43 - protein, 12 aa, Artificial Sequence
  Synthetic Peptide
SLGPQGIWGQFN